(19) United States
(12) Patent Application Publication
(10) Pub. No.: US 2025/0171741 A1
(43) Pub. Date: May 29, 2025

(54) METHODS AND SYSTEMS FOR DIFFERENTIATION OF CELLS USING OPTOGENETICS

(71) Applicant: Prolific Machines Inc., Emeryville, CA (US)

(72) Inventors: Deniz KENT, Oakland, CA (US); Maximiliaan HUISMAN, Oakland, CA (US); Declan JONES, Oakland, CA (US); Victor JONES, Berkeley, CA (US); Monique HOOLEY, Oakland, CA (US); Emily GALE, Berkeley, CA (US)

(21) Appl. No.: 18/962,717

(22) Filed: Nov. 27, 2024

Related U.S. Application Data
(63) Continuation of application No. PCT/US2023/067784, filed on Jun. 1, 2023.
(60) Provisional application No. 63/347,780, filed on Jun. 1, 2022.

(51) Int. Cl. C12N 5/077; C12N 9/22; C12N 15/85

(52) U.S. Cl. CPC C12N 5/0653; C12N 9/22; C12N 15/85; C12N 2506/1307; C12N 2800/107; C12N 2800/90; C12N 2830/002

(57) ABSTRACT

Provided herein are methods and systems for differentiating cells in a population of cells into a desired cell lineage. In some embodiments, the methods and systems use light (e.g., optogenetics) to control differentiation of cells into a desired cell lineage. In some cases, the methods and systems involve differentiating cells in a suspension culture for large-scale production of cells of a desired cell lineage.

### CROSS-REFERENCE

This application is a continuation of International Application No. PCT/US2023/067784, filed Jun. 1, 2023, which claims the benefit of U.S. Provisional Application No. 63/347,780, filed Jun. 1, 2022, each of which is incorporated herein by reference in its entirety.

### BACKGROUND

Transcription factors can regulate the differentiation of (e.g., stem) cells into different cell types. Expression of transcription factors such as MyoD can result in cells differentiating into muscle cells, while expression of other transcription factors such as PPARγ can cause cells to differentiate into adipocytes or adipocyte-like cells. Differentiation of a population of (e.g., stem) cells into a single cell lineage, for example with high efficiency and temporal precision, can be difficult.

### SUMMARY

Methods and systems described herein fulfill an unmet need for efficient differentiation of a population of cells (e.g., stem cells) into a specified or desired cell lineage (or cell type), for example, using optogenetics.

In one aspect, a method of differentiating at least one cell of a population of cells into a desired cell lineage is provided, the method comprising: (a) providing or obtaining the population of cells in a suspension culture; and (b) controlling differentiation of the at least one cell of the population of cells with light, thereby differentiating the at least one cell into a desired cell lineage. In some cases, the controlling differentiation of (b) comprises illuminating the at least one cell with light at a first wavelength. In some cases, the controlling differentiation of (b) comprises removing light at a first wavelength from the at least one cell. In some cases, the at least one cell is genetically engineered to contain an exogenous nucleic acid comprising a nucleic acid sequence encoding for at least one transcription factor, at least one differentiation factor, or both, that effect differentiation into the desired cell lineage. In some cases, the expression of the at least one transcription factor, the at least one differentiation factor, or both, is induced by the illuminating or the removing light. In some cases, the exogenous nucleic acid comprises at least one promoter operably linked to the at least one transcription factor, the at least one differentiation factor, or both. In some cases, the at least one cell is genetically engineered to contain an exogenous nucleic acid comprising a nucleic acid sequence encoding for at least one light-controllable transcriptional regulator. In some cases, the promoter is an inducible promoter. In some cases, the at least one light-controllable transcriptional regulator is a light-controllable transcriptional activator. In some cases, the light-controllable transcriptional activator comprises a transcriptional activator fused to a light-controllable domain. In some cases, the illuminating induces the light-controllable transcriptional activator to bind to and activate the inducible promoter, thereby causing expression of the at least one transcription factor, the at least one differentiation factor, or both. In some cases, the removing light induces the light-controllable transcriptional activator to bind to and activate the inducible promoter, thereby causing expression of the at least one transcription factor, the at least one differentiation factor, or both. In some cases, the promoter is a constitutive promoter. In some cases, the at least one light-controllable transcriptional regulator is a light-controllable transcriptional repressor. In some cases, the light-controllable transcriptional repressor comprises a transcriptional repressor fused to a light-controllable domain. In some cases, the illuminating induces the light-controllable transcriptional repressor to dissociate from the constitutive promoter, thereby causing expression of the at least one transcription factor, the at least one differentiation factor, or both. In some cases, the removing light induces the light-controllable transcriptional repressor to dissociate from the constitutive promoter, thereby causing expression of the at least one transcription factor, the at least one differentiation factor, or both. In some cases, the exogenous nucleic acid further comprises a blocking sequence downstream of the at least one promoter which, when present, blocks expression of the at least one first transcription factor, the at least one differentiation factor, or both. In some cases, the at least one cell further comprises a nucleic acid sequence encoding at least one light-controllable recombinase. In some cases, the at least one light-controllable recombinase is a light-activatable recombinase. In some cases, the light-activatable recombinase comprises a recombinase or a portion thereof fused to a light-activatable domain. In some cases, the blocking sequence is flanked by recombinase recognition sites that are recognized by the at least one light-controllable recombinase. In some cases, the illuminating activates the light-controllable recombinase, thereby resulting in excision of the blocking sequence and inducing expression of the at least one transcription factor, the at least one differentiation factor, or both. In some cases, the at least one cell is a stem cell. In some cases, the stem cell is a pluripotent stem cell or a multipotent stem cell. In some cases, the at least one cell is a fibroblast cell. In some cases, the at least one cell is a human cell, a bovine cell, or a mouse cell. In some cases, the desired cell lineage is selected from the group consisting of: an adipocyte, a myocyte, and a chondrocyte. In some cases, the at least one transcription factor is selected from the group consisting of: PPARγ, CEBP alpha, MYOD, MYOG, Myf5, MRF4, HEYL, KLF4, PAX3, SOX9, SOX5, SOX6, and any combination thereof. In some cases, the illuminating further comprises illuminating a plurality of cells of the population of cells with light to differentiate each of the plurality of cells into the desired cell lineage; or the removing further comprises removing light from the population of cells to differentiate each of the plurality of cells into the desired cell lineage. In some cases, the suspension culture has a volume of at least 100 milliliters (mL). In some cases, the suspension culture is contained within a bioreactor vessel. In some cases, the bioreactor vessel has a total volume of at least 100 milliliters (mL). In some cases, the population of cells are grown on the surface of a microcarrier. In some cases, the microcarrier is coated with one or more extracellular matrix components.

In another aspect, a system for differentiating a population of cells is provided, the system comprising: (a) a population of cells in suspension culture, wherein at least one cell of the population of cells is engineered to contain an exogenous nucleic acid comprising a nucleic acid sequence encoding for at least one transcription factor, at least one differentiation factor, or both, that effects differentiation into a desired cell lineage, wherein expression of the at least one transcription factor, the at least one differentiation factor, or both, is controlled by light; and (b) one or more light source configured to illuminate at least one cell of the population of cells with light at a first wavelength. In some cases, the exogenous nucleic acid comprises at least one promoter operably linked to the at least one transcription factor, the at least one differentiation factor, or both. In some cases, the at least one cell is genetically engineered to contain an exogenous nucleic acid comprising a nucleic acid sequence encoding for at least one light-controllable transcriptional regulator. In some cases, the promoter is an inducible promoter. In some cases, the at least one light-controllable transcriptional regulator is a light-controllable transcriptional activator. In some cases, the light-controllable transcriptional activator comprises a transcriptional activator fused to a light-controllable domain. In some cases, the light-controllable transcriptional activator, upon illumination by light at the first wavelength, binds to and activates the inducible promoter, thereby causing expression of the at least one transcription factor, the at least one differentiation factor, or both. In some cases, the light-controllable transcriptional activator, upon removal of light at the first wavelength, binds to and activates the inducible promoter, thereby causing expression of the at least one transcription factor, the at least one differentiation factor, or both. In some cases, the promoter is a constitutive promoter. In some cases, the at least one light-controllable transcriptional regulator is a light-controllable transcriptional repressor. In some cases, the light-controllable transcriptional repressor comprises a transcriptional repressor fused to a light-controllable domain. In some cases, the light-controllable transcriptional repressor, upon illumination by light at the first wavelength, dissociates from the constitutive promoter, thereby causing expression of the at least one transcription factor, the at least one differentiation factor, or both. In some cases, the light-controllable transcriptional repressor, upon removal of light at the first wavelength, dissociates from the constitutive promoter, thereby causing expression of the at least one transcription factor, the at least one differentiation factor, or both. In some cases, the exogenous nucleic acid further comprises a blocking sequence downstream of the at least one promoter which, when present, blocks expression of the at least one transcription factor, the at least one differentiation factor, or both. In some cases, the at least one cell of the population of cells further comprises a nucleic acid sequence encoding at least one light-controllable recombinase. In some cases, the at least one light-controllable recombinase is a light-activatable recombinase. In some cases, the light-activatable recombinase comprises a recombinase fused to a light-activatable domain. In some cases, the blocking sequence is flanked by recombinase recognition sites that are recognized by the at least one light-controllable recombinase. In some cases, the blocking sequence is configured to be excised by the at least one light-controllable recombinase, thereby inducing expression of the at least one transcription factor. In some cases, the at least one cell is a stem cell. In some cases, the stem cell is a pluripotent stem cell or a multipotent stem cell. In some cases, the at least one cell is a fibroblast cell. In some cases, the at least one cell is a human cell, a bovine cell, or a mouse cell. In some cases, the desired cell lineage is selected from the group consisting of: an adipocyte, a myocyte, and a chondrocyte. In some cases, the at least one transcription factor is selected from the group consisting of: PPARγ, CEBP alpha, MYOD, MYOG, Myf5, MRF4, HEYL, KLF4, PAX3, SOX9, SOX5, SOX6, and any combination thereof. In some cases, the system further comprises a plurality of microcarriers, wherein the population of cells are grown on the surface of the plurality of microcarriers. In some cases, the plurality of microcarriers are coated with one or more extracellular matrix components. In some cases, the one or more light source comprises one or more light-emitting diodes (LEDs). In some cases, the one or more light source comprises one or more lasers. In some cases, the one or more light source comprises an incandescent light source.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A and 1B depict non-limiting examples of a system for differentiating cells into a desired cell lineage using a light-dimerizing domain coupled to a transcriptional activator, in accordance with embodiments herein.

FIGS. 2A and 2B depict non-limiting examples of a system for differentiating cells into a desired cell lineage using a light-dissociating domain coupled to a transcriptional activator, in accordance with embodiments herein.

FIGS. 3A and 3B depict non-limiting examples of a system for differentiating cells into a desired cell lineage using a light-dimerizing domain coupled to a transcriptional repressor, in accordance with embodiments herein.

FIGS. 4A and 4B depict non-limiting examples of a system for differentiating cells into a desired cell lineage using a light-dissociating domain coupled to a transcriptional repressor, in accordance with embodiments herein.

FIG. 5 depicts a non-limiting example of a schematic of a nucleic acid sequence for expression of a transcription factor that is suitable for use with the methods and systems described herein.

FIG. 6 depicts a non-limiting example of an inducible recombinase system that is suitable for use with the methods and systems described herein.

FIGS. 7A and 7B depict non-limiting examples of constructs suitable for use with the methods and systems described herein.

FIGS. 8A and 8B depict non-limiting examples of constructs suitable for use with the methods and systems described herein.

FIGS. 9A and 9B depict data demonstrating accumulation of lipid droplets in fibroblasts grown in two-dimensional culture that constitutively express adipogenic transcription factors, in accordance with the methods and systems described herein.

FIG. 10 depicts data demonstrating accumulation of lipid droplets in fibroblasts grown in suspension culture that constitutively express adipogenic transcription factors, in accordance with the methods and systems described herein.

FIGS. 11A and 11B depict data demonstrating activation of a light-activatable recombinase using light, in accordance with the methods and systems described herein.

FIG. 12 depicts data demonstrating accumulation of lipid droplets in fibroblasts grown in two-dimensional culture that express adipogenic transcription factors under the control of a constitutively expressed recombinase, in accordance with the methods and systems described herein.

FIGS. 13A and 13B depict data demonstrating accumulation of lipid droplets in fibroblasts grown in two-dimensional culture that express adipogenic transcription factors under the control of a constitutively expressed recombinase, in accordance with the methods and systems described herein.

FIG. 14 depicts data demonstrating accumulation of lipid droplets in fibroblasts grown in two-dimensional culture that express adipogenic transcription factors under the control of a light-activatable recombinase, in accordance with the methods and systems described herein.

FIGS. 15A and 15B depict data demonstrating accumulation of lipid droplets in fibroblasts grown in two-dimensional culture that express adipogenic transcription factors under the control of a light-activatable recombinase, in accordance with the methods and systems described herein.

FIG. 16 depicts data demonstrating accumulation of lipid droplets in fibroblasts grown in suspension culture that express adipogenic transcription factors under the control of a light-activatable recombinase, in accordance with the methods and systems described herein.

FIG. 17 depicts data demonstrating accumulation of lipid droplets in fibroblasts grown in suspension culture that express adipogenic transcription factors under the control of a light-activatable recombinase, in accordance with the methods and systems described herein.

FIG. 18 depicts data from a control experiment, in accordance with the methods and systems described herein.

FIGS. 19A and 19B depict data demonstrating expression of genes associated with adipogenesis in fibroblasts grown in suspension culture that have been differentiated to towards adipocyte-like cells using the methods and systems described herein.

### DETAILED DESCRIPTION

The developmental fate of a cell (e.g., a stem cell) can be determined by expressing particular transcription factors in the cell that set in motion a developmental program leading to its differentiation into a particular cell type. However, control (e.g., temporal control) over the differentiation of a population of cells (e.g., stem cells) into one or more specified or desired cell lineages or cell types in a controlled manner can be difficult using existing technology. For instance, differentiation of stem cells into a specified or desired cell type (e.g., wherein the specified or desired cell type comprises all or a portion of a three-dimensional tissue) can require high temporal or spatiotemporal precision. As described herein, controlling the (e.g., temporal) expression of transcription factors in stem cells can allow for (e.g., simultaneous and/or patterned) differentiation of a single population of stem cells into a population of cells comprising one or more cell types. For example, methods and systems disclosed herein can be used to differentiate a single population of (e.g., stem) cells into a single specified or desired cell type, for instance, while minimizing or preventing differentiation of all or a portion of the single population of cells into a cell type other than the specified or desired cell type. The methods and systems provided herein generally use optogenetics to control differentiation of cells (e.g., stem cells) into a desired cell lineage. In some instances, the methods and systems provided herein use light-controllable transcriptional regulators (e.g., light-controllable transcriptional activators, light-controllable transcriptional repressors, and the like) that are controlled by light (e.g., either by illuminating the (e.g., stem) cell with light at a particular wavelength or wavelength range, or by removing light of a particular wavelength or wavelength range from the (e.g., stem) cell) to control expression of transcription factor(s) and/or differentiation factor(s) leading to differentiation of a (e.g., stem) cell into a desired cell lineage. In other instances, the methods and systems disclosed herein use light-controllable recombinases (e.g., light-activatable recombinases) that are controlled by light in order to differentiate a (e.g., stem) cell into a desired cell lineage.

Generally, the methods and systems provided herein use optogenetics to activate a desired differentiation pathway(s) in a (e.g., stem) cell. The use of optogenetics to differentiate cells is relatively inexpensive compared to traditional methods (e.g., that use expensive chemical factors). Furthermore, by not requiring the addition of exogenous chemical factors, the methods and systems provided herein are a safe alternative to traditional methods, especially when the differentiated cells are produced for downstream use by an individual (e.g., for consumption by an individual or for therapeutic use). Additionally, the use of light to differentiate cells allows individual cells or a subset of a population of cells to be illuminated with light, rather than the entire population of cells, as is typical with the addition of chemical factors. This provides precise temporal and spatial control of differentiation in a cell population. Furthermore, in some embodiments, the methods and systems provided herein are tunable, as light can easily be supplied or removed to induce or block expression of differentiation pathways (e.g., such as when light-inducible promoters are used). In addition, the amount of gene expression can be controlled, for example, by adjusting the intensity of light.

Advantageously, the methods and systems provided herein are capable of differentiating (e.g., stem) cells in suspension culture into a desired cell lineage(s). The use of suspension culture allows for large-scale production of desired cell types, which is not achievable with cells in adherent cultures (e.g., in a well of a tissue culture plate). Thus, the methods and systems provided herein are particularly advantageous when the large-scale production of a particular type or lineage of cell is desired.

In some embodiments, the methods and systems provided herein use optogenetics for (e.g., temporally and/or spatially) controlling differentiation of cells into a desired cell lineage or cell type. For instance, in some embodiments, a promoter regulating (e.g., expression of) one or more transcription factor and/or differentiation factor (e.g., involved in a differentiation pathway) may be controlled by light. In some cases, the methods and systems involve the use of light-controllable transcriptional regulators which can either bind to a promoter (or a region adjacent to or near a promoter), or dissociate from a promoter (or a region adjacent to or near a promoter), to activate expression of the one or more transcription factor and/or differentiation factor. In some cases, the light-controllable transcriptional regulator is a transcriptional regulator fused to a light-controllable domain (e.g., an optical switch). In some cases, the light-controllable transcriptional regulator is a light-controllable transcriptional activator. The light-controllable transcriptional activator may be a transcriptional activator fused to a light-controllable domain (e.g., an optical switch). In some cases, the light-controllable transcriptional regulator is a light-controllable transcriptional repressor. The light-controllable transcriptional repressor may be a transcriptional repressor fused to a light-controllable domain. Advantageously, such methods and systems do not lead to permanent activation of a differentiation pathway, and the differentiation pathway may be turned on or off simply by supplying or removing light, as described herein.

In some embodiments, the methods and systems provided herein use one or more light-controllable recombinases. A recombinase recognizes a specific DNA sequence, and if there are two recognition sequences in the proper arrangement, it can excise or flip the orientation of the DNA between the two sites. By briefly activating the recombinase, a permanent change can be made to the DNA, which offers the prospect of permanently switching on the differentiation genes with only a short activation phase. Such methods and systems may lead to, e.g., permanent activation of a differentiation program.

Methods and systems for differentiating a population of (e.g., stem) cells (e.g., a population of individual (e.g., stem) cells), for example using a suspension cell culture technique, are disclosed herein. In some cases, a system described herein can be configured to (and/or used to) differentiate a first population of (e.g., stem) cells into cells of a desired cell lineage (e.g., wherein the cells of the desired cell lineage comprises one or more phenotype(s), gene expression profile(s), and/or epigenetic state(s) that is/are different than that of the undifferentiated cells). In some cases, a system described herein can be used to perform a method described herein. In some cases, systems described herein can be useful in facilitating differentiation of a population of cells to a specified or desired cell type, for example, by establishing and/or maintaining conditions necessary or beneficial for the differentiation of all or a portion of the population of cells to the specified or desired cell type. In some cases, systems described herein can be useful in facilitating differentiation of a population of cells to a specified or desired cell type, for example, by establishing and/or maintaining conditions necessary to prevent or beneficial in preventing differentiation of all or a portion of the population of cells to a cell type different than the specified or desired cell type (e.g., a cell type comprising an additional phenotype, gene expression profile, and/or epigenetic state that is/are different than that of the specified or desired cell type). The methods and systems, in some cases, include the use of light-controllable transcriptional regulators and/or light-controllable recombinases. Provided herein are methods for differentiating a population of (e.g., stem) cells. The methods may comprise providing or obtaining a population of (e.g., stem) cells (e.g., engineered (e.g., stem) cells), and controlling differentiation of at least one (e.g., stem) cell of the population of (e.g., stem) cells into a desired cell lineage with light. The differentiating, in some cases, may involve illuminating the at least one (e.g., stem) cell of the population of (e.g., stem) cells with light at a first wavelength to differentiate the at least one (e.g., stem) cell into a desired cell lineage. The differentiating, in some cases, may involve removing light of a particular wavelength (or removing light completely) to differentiate the at least one (e.g., stem) cell into a desired cell lineage. In some cases, population of (e.g., stem) cells are in a suspension culture (e.g., in a bioreactor)

Expression of at least one transcription factor and/or differentiation factor in a cell may result in differentiation of the cell into a particular cell type. Any transcription factor or combination of transcription factors, and/or any differentiation factor or combination of differentiation factors that, when expressed, leads to differentiation of a cell to a desired cell lineage is contemplated herein. In some cases, differentiation of a cell into a specified or desired cell lineage or cell type (e.g., adipocyte, myocyte, or chondrocyte) can be induced by a transcription factor, of which non-limiting examples include PPAR gamma, CEBP alpha, MYOD, MYOG, Myf5, MRF4, HEYL, KLF4, PAX, SOX9, SOX5, SOX6, or any combination thereof. In a non-limiting example, when differentiation of a cell into a fat cell (e.g., adipocyte) is desired, the cell may be induced to express a transcription factor such as, but not limited to, PPAR gamma, and/or CEBP alpha. In another non-limiting example, when differentiation of a cell into a muscle cell (e.g., myocyte) is desired, the cell may be induced to express a transcription factor such as, but not limited to, MYOD, MYOG, Myf5, MRF4, HEYL, KLF4, and/or PAX3. In another non-limiting example, when differentiation of a cell into a cartilage cell (e.g., chondrocyte) is desired, the cell may be induced to express a transcription factor such as, but not limited to, SOX9, SOX5, and/or SOX6. In some embodiments, the at least one transcription factor is selected from the group consisting of: PPAR gamma, CEBP alpha, MYOD, MYOG, and a combination thereof. In some cases, the at least one differentiation factor may include at least one chromatin remodeling factor. In some cases, the chromatin remodeling factor may be SMARCD3 and/or JMJD3. In some cases, expression of the transcription factor and/or differentiation factor may be regulated e.g., by light.

In certain aspects, the methods described herein may involve expressing at least one, at least two, at least three, at least four, at least five, at least six, at least seven, at least eight, at least nine, or at least ten transcription factors and/or differentiation factors (e.g., chromatin remodeling factors) in the (e.g., stem) cell (e.g., to differentiate the (e.g., stem) cell into a desired cell lineage).

In some aspects, a cell population (e.g., an engineered cell population) described herein may comprise at least one exogenous nucleic acid. The exogenous nucleic acid may comprise a sequence encoding any transcription factor and/or other differentiation factor (e.g., chromatin remodeling factors) as described herein. The sequence encoding the transcription factor and/or other differentiation factor may be operably linked to a promoter. The promoter sequence may be constitutively active. In an alternate embodiment, a promoter sequence may be conditionally active. For instance, a conditionally active promoter sequence may be regulatable or inducible, e.g., by light. In some aspects, the cells described herein comprise at least two exogenous nucleic acids. In some aspects, the cells described herein comprise at least three, at least four, at least five, or more, exogenous nucleic acids. Each exogenous nucleic acid may comprise at least one nucleic acid sequence encoding one or more transcription factor(s) and/or differentiation factor(s). The nucleic acid sequence(s) encoding the transcription factor(s) and/or differentiation factor(s) may be operably linked to one or more promoters (e.g., constitutive, inducible), as described herein. In some cases, when more than one transcription factor and/or differentiation factor is used to differentiate a cell to a desired cell lineage, expression of each of the more than one transcription factor and/or differentiation factor may be under the control of the same, single promoter. In such cases, each gene encoding the transcription factor and/or differentiation factor may be combined into a single, bi-cistronic or multi-cistronic transcript. The single transcript may either encode self-cleaving 2A peptides between each separate protein, or may contain internal ribosome entry sites (IRES), or a combination thereof. In other cases, expression of the transcription factor(s) and/or differentiation factor(s) may be under the control of different promoters.

In some cases, at least one (e.g., stem) cell of a (e.g., stem) cell population described herein may be genetically engineered to contain an exogenous nucleic acid comprising a nucleic acid sequence encoding at least one light-controllable transcriptional regulator. In some cases, the at least one light-controllable transcriptional regulator is a light-controllable transcriptional activator. The light-controllable transcriptional activator may be a transcriptional activator that is fused to or otherwise associated with a light-controllable domain, such as an optical switch (e.g., as described herein). In some cases, the at least one light-controllable transcriptional regulator may be a light-controllable transcriptional repressor. The light-controllable transcriptional repressor may be a transcriptional repressor that is fused to or otherwise associated with a light-controllable domain, such as an optical switch (e.g., as described herein).

In various aspects, the light-controllable transcriptional regulator is a light-controllable transcriptional activator. In one embodiment, the light-controllable transcriptional activator, upon illumination with light (e.g., at a particular wavelength or wavelength range), binds to or otherwise associates with a promoter sequence (e.g., an inducible promoter), thereby inducing expression of one or more downstream genes (e.g., one or more transcription factor and/or differentiation factor, as described herein).

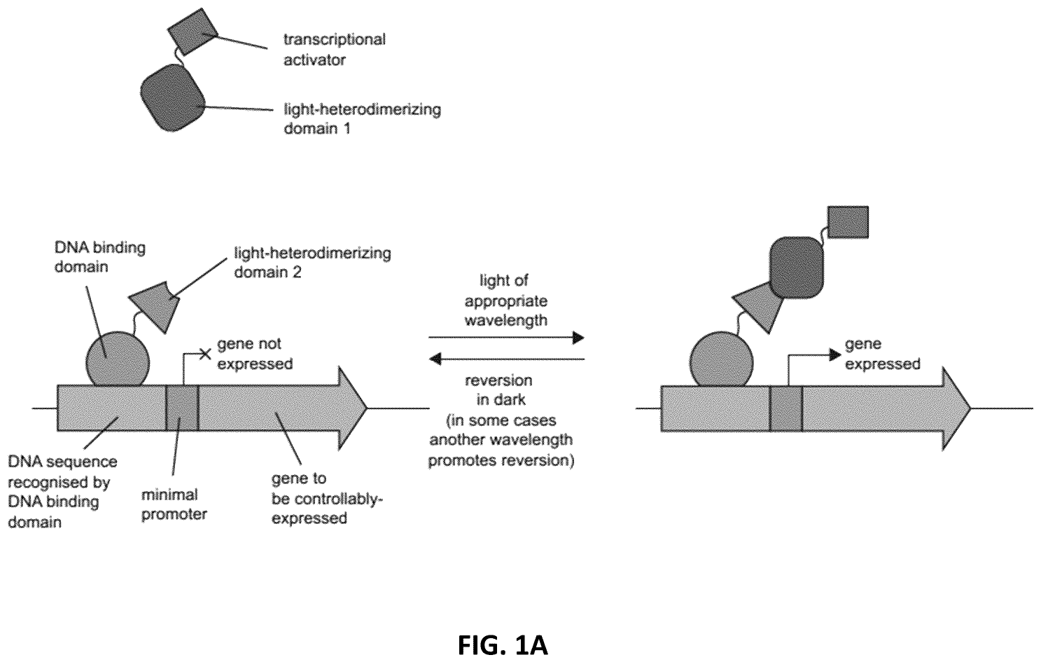

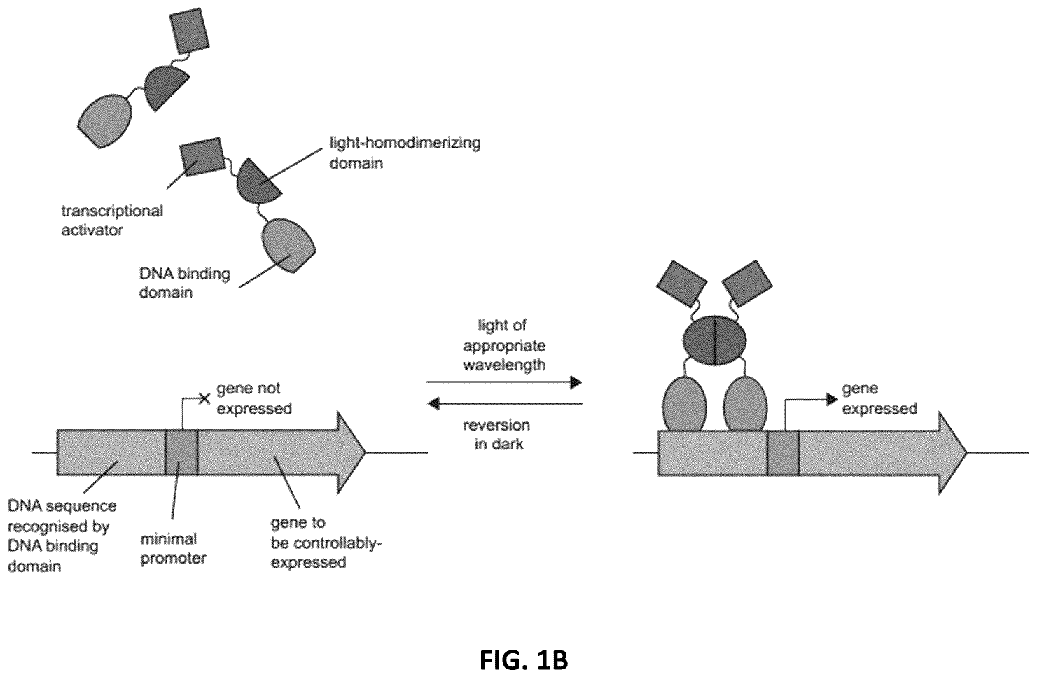

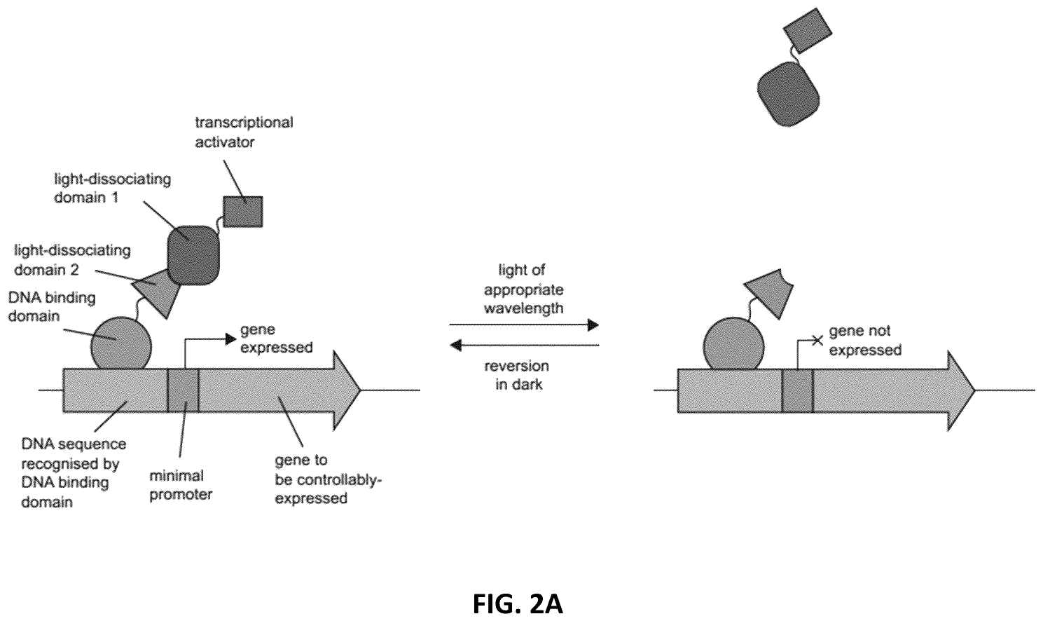

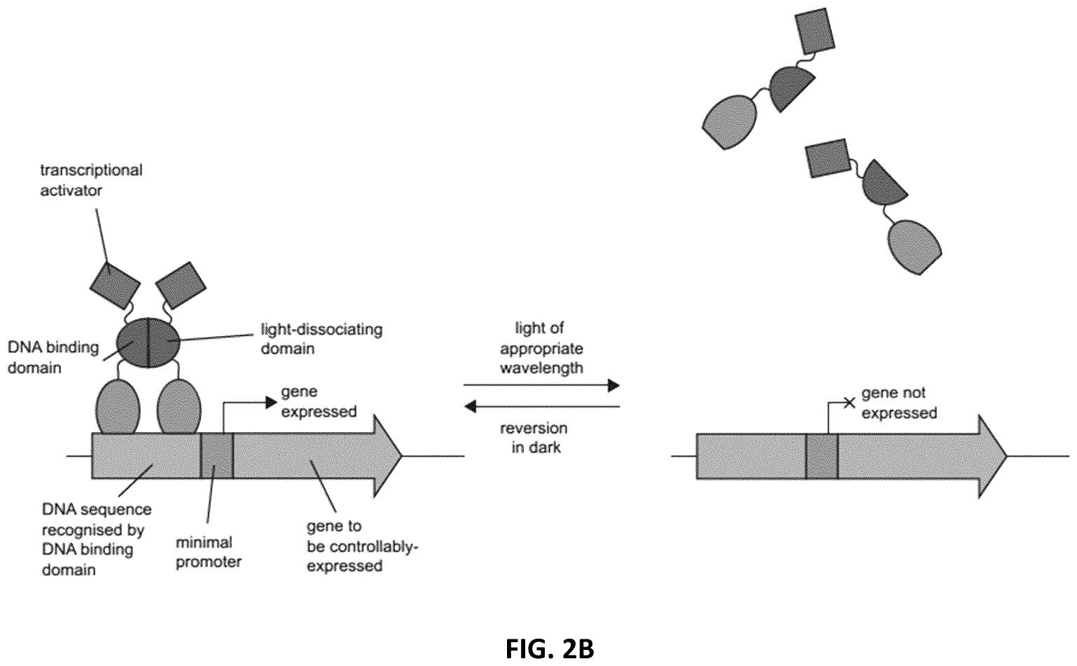

FIGS. 1A, 1B, 2A, and 2B depict various embodiments that use a light-controllable activator to control expression of one or more transcription factor and/or differentiation factor. As shown in FIG. 1A, in one embodiment, a transcriptional activator is fused to a first light-heterodimerizing domain (e.g., light-heterodimerizing domain 1). A second light-heterodimerizing domain (e.g., light-heterodimerizing domain 2), which is capable of dimerizing with the first light-heterodimerizing domain, is fused to a DNA-binding domain. The DNA-binding domain binds to a DNA sequence within or near the promoter sequence. In the absence of light, the first and second light-heterodimerizing domains are not associated and expression of the transcription factor(s) and/or differentiation factor(s) is turned off. Upon illumination with light (e.g., at an appropriate wavelength or wavelength range), the first and second light-heterodimerizing domains heterodimerize, bringing the transcriptional activator into close contact with the inducible promoter. The transcriptional activator then induces activity of the inducible promoter and the transcription factor(s) and/or differentiation factor(s) are expressed, leading to differentiation into a desired cell lineage. In this scenario, those cells that are illuminated with light differentiate to a desired cell lineage, whereas those cells that are not illuminated with light, or not illuminated with light at the appropriate wavelength, remain undifferentiated.

In another embodiment, as depicted in FIG. 1B, a transcriptional activator is fused to a light-homodimerizing domain and a DNA-binding domain. In this scenario, the DNA-binding domain, upon homodimerizing with another DNA-binding domain, binds to a DNA sequence within or near the promoter sequence. Upon illumination with light (e.g., at an appropriate wavelength or wavelength range), the light-homodimerizing domains homodimerize, which brings two DNA-binding domains into close contact such that they homodimerize. Upon homodimerizing, the DNA-binding domain binds to the promoter sequence bringing the transcriptional activator into close contact with the inducible promoter. The transcriptional activator then induces activity of the inducible promoter and the transcription factor(s) and/or differentiation factor(s) are expressed, leading to differentiation into a desired cell lineage. In this scenario, those cells that are illuminated with light differentiate to a desired cell lineage, whereas those cells that are not illuminated with light, or not illuminated with light at the appropriate wavelength, remain undifferentiated.

In various aspects, the light-controllable transcriptional activator, in the absence of light or in the absence of light at a particular wavelength, binds to or associates with a promoter sequence (e.g., an inducible promoter), thereby inducing expression of one or more downstream genes (e.g., one or more transcription factor and/or differentiation factor, as described herein). In the presence of light, the light-controllable transcriptional activator does not bind to or dissociates from the promoter sequence (e.g., an inducible promoter), thereby preventing or reducing expression of one or more downstream genes (e.g., one or more transcription factor and/or differentiation factor, as described herein).

As shown in FIG. 2A, in one embodiment, a transcriptional activator is fused to a first light-dissociating domain (e.g., light-dissociating domain 1). A second light-dissociating domain (e.g., light-dissociating domain 2) is fused to a DNA-binding domain. The DNA-binding domain binds to a DNA sequence within or near the promoter sequence. In the absence of light, the first and second light-dissociating domains are associated, bringing the transcriptional activator in close contact with the inducible promoter, and expression of the transcription factor(s) and/or differentiation factor(s) is turned on. Upon illumination with light (e.g., at an appropriate wavelength or wavelength range), the first and second light-dissociating domains dissociate, releasing the transcriptional activator from the inducible promoter thereby turning off expression of the transcription factor(s) and/or differentiation factor(s). In this scenario, those cells that are illuminated with light remain undifferentiated, whereas those cells that are not illuminated with light, or not illuminated with light at the appropriate wavelength, differentiate into a desired cell lineage.

In another embodiment, as depicted in FIG. 2B, a transcriptional activator is fused to a light-dissociating domain and a DNA-binding domain. In this scenario, the DNA-binding domain, upon homodimerizing with another DNA-binding domain, binds to a DNA sequence within or near the promoter sequence. In the absence of light, the light-dissociating domain is homodimerized with another light-dissociating domain, allowing the DNA-binding domain to homodimerize and bind the inducible promoter, and bringing the transcriptional activator into close contact with the inducible promoter. The transcriptional activator then induces activity of the inducible promoter and the transcription factor(s) and/or differentiation factor(s) are expressed, leading to differentiation into a desired cell lineage. Upon illumination with light (e.g., at an appropriate wavelength or wavelength range), the light-dissociating domains dissociate, which releases the transcriptional activator from the inducible promoter and turns off expression of the transcription factor(s) and/or differentiation factor(s). In this scenario, those cells that are illuminated with light remain undifferentiated, whereas those cells that are not illuminated with light, or not illuminated with light at the appropriate wavelength, differentiate into a desired cell lineage.

In various aspects, the light-controllable transcriptional regulator is a light-controllable transcriptional repressor. In one embodiment, the light-controllable transcriptional repressor, upon illumination with light (e.g., at a particular wavelength or wavelength range), binds to or otherwise associates with a promoter sequence (e.g., a constitutive promoter, or a sequence between a constitutive promoter and a transcriptional start site), thereby repressing the constitutive promoter and preventing or reducing expression of one or more downstream genes (e.g., one or more transcription factor and/or differentiation factor, as described herein). In the absence of light, the light-controllable transcriptional repressor does not bind to or dissociates from the promoter sequence (e.g., a constitutive promoter, or a sequence between a constitutive promoter and a transcriptional start site), thereby allowing expression of one or more downstream genes (e.g., one or more transcription factor and/or differentiation factor, as described herein). In such instances, those (e.g., stem) cells that are illuminated with light remain undifferentiated, whereas those (e.g., stem) cells that are not illuminated with light differentiate into the desired cell lineage.

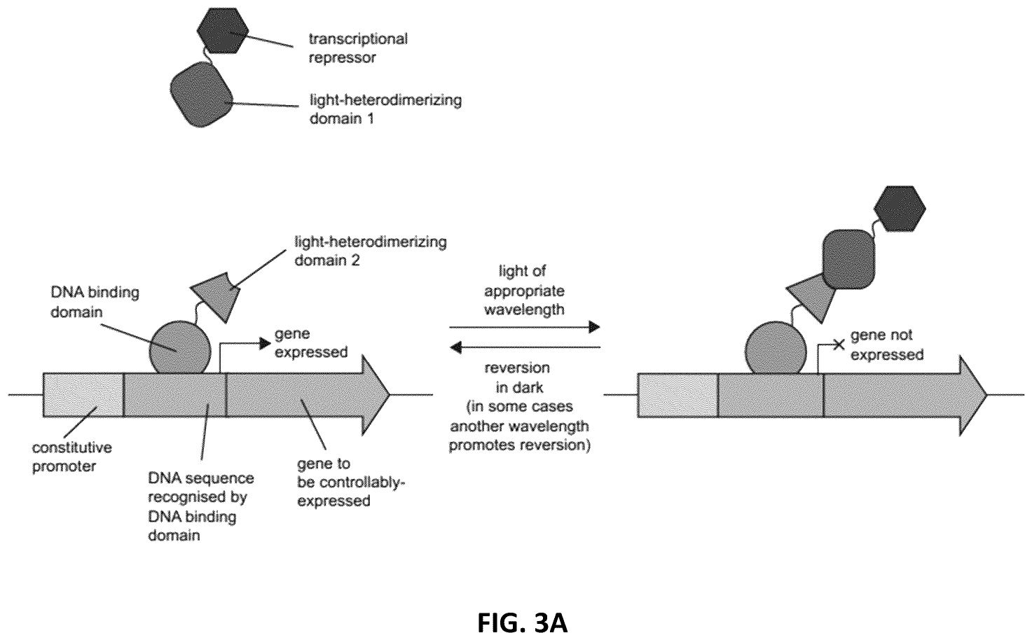

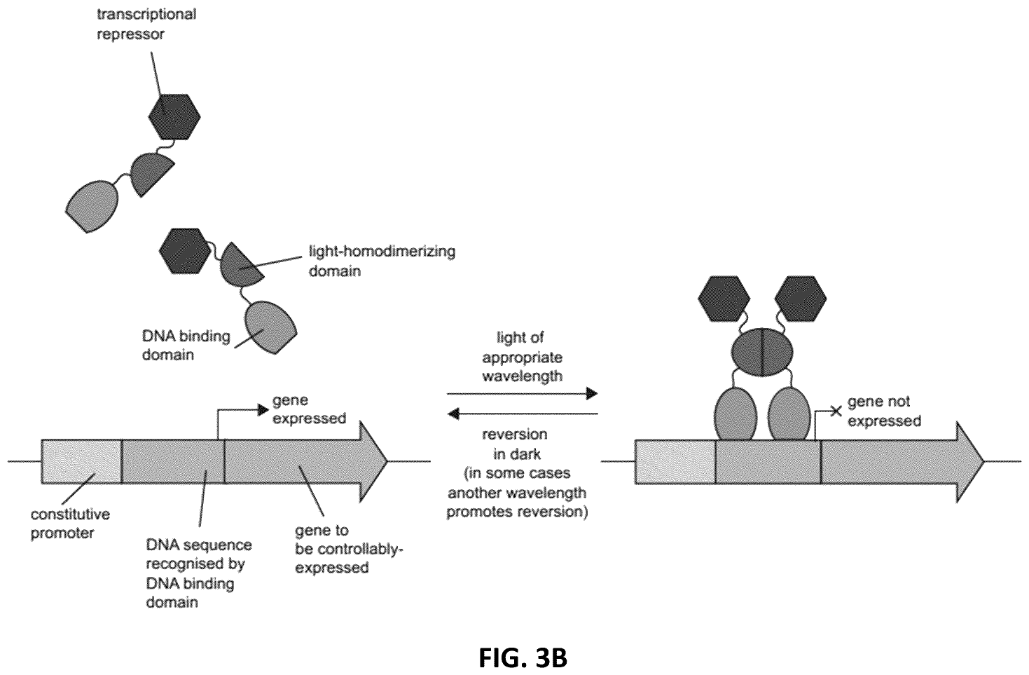

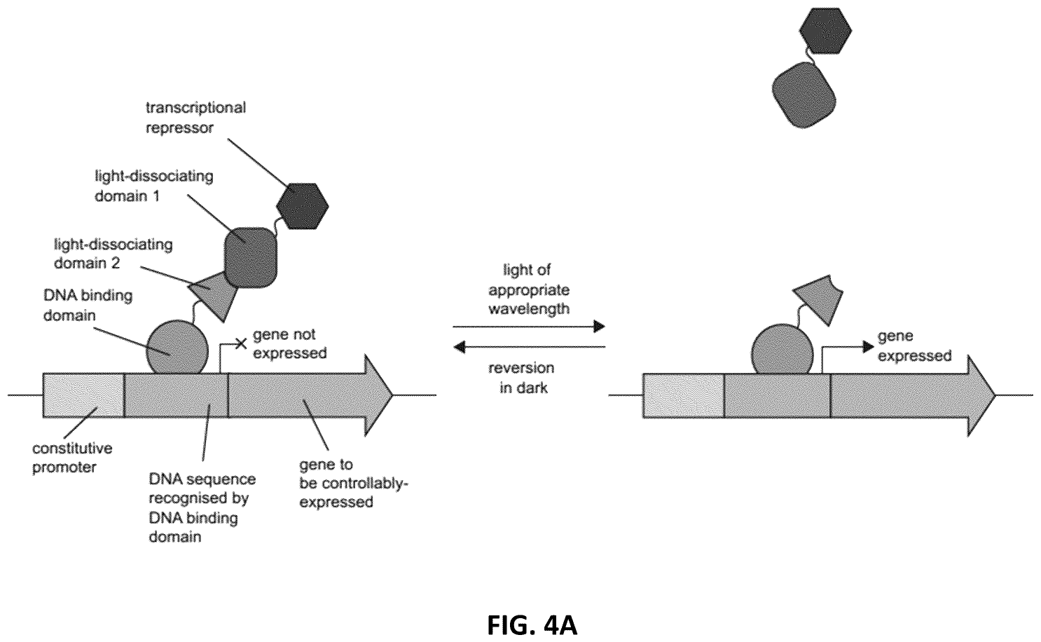

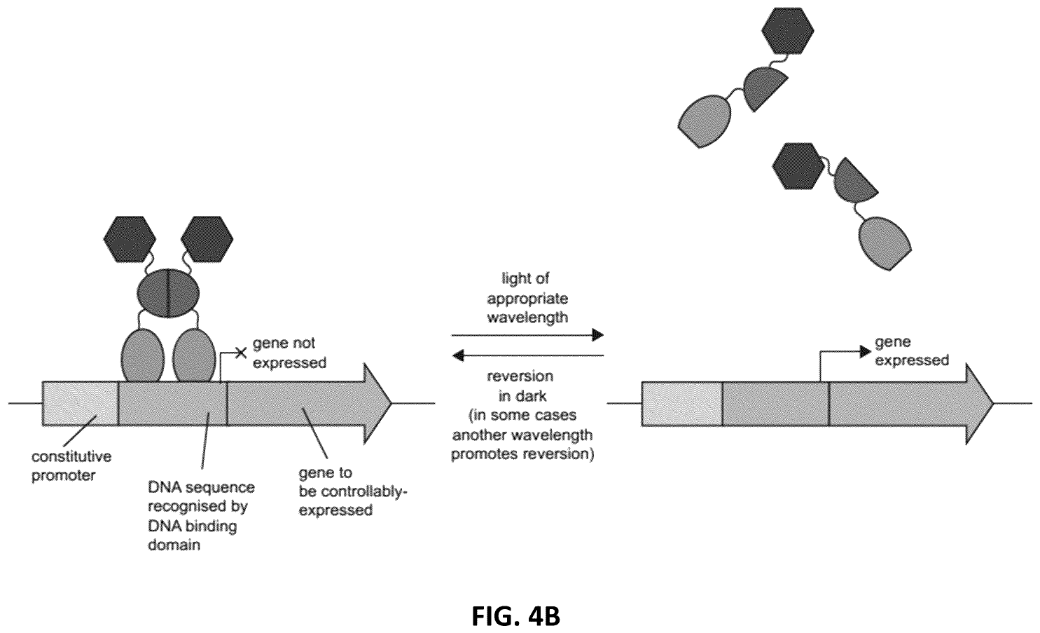

FIGS. 3A, 3B, 4A, and 4B depict various embodiments that use a light-controllable transcriptional repressor to control expression of one or more transcription factor and/or differentiation factor. As shown in FIG. 3A, in one embodiment, a transcriptional repressor is fused to a first light-heterodimerizing domain (e.g., light-heterodimerizing domain 1). A second light-heterodimerizing domain (e.g., light-heterodimerizing domain 2), which is capable of dimerizing with the first light-heterodimerizing domain, is fused to a DNA-binding domain. The DNA-binding domain binds to a DNA sequence within or near the promoter sequence. In the absence of light, the first and second light-heterodimerizing domains are not associated and expression of the transcription factor(s) and/or differentiation factor(s) is turned on (e.g., because the genes encoding the transcription factor(s) and/or differentiation factor(s) are under the control of a constitutive promoter). Upon illumination with light (e.g., at an appropriate wavelength or wavelength range), the first and second light-heterodimerizing domains dimerize, bringing the transcriptional repressor into close contact with the promoter sequence or a sequence between the promoter and the transcriptional start site. The transcriptional repressor blocks activity of the constitutive promoter and expression of the transcription factor(s) and/or differentiation factor(s) is turned off. In this scenario, those cells that are illuminated with light remain undifferentiated, whereas those cells that are not illuminated with light, or not illuminated with light at the appropriate wavelength, differentiate into a desired cell lineage.

In another embodiment, as depicted in FIG. 3B, a transcriptional repressor is fused to a light-homodimerizing domain and a DNA-binding domain. In this scenario, the DNA-binding domain, upon homodimerizing with another DNA-binding domain, binds to a DNA sequence within or near the promoter sequence. In the absence of light, the first and second light-homodimerizing domains are not associated and expression of the transcription factor(s) and/or differentiation factor(s) is turned on (e.g., because the genes encoding the transcription factor(s) and/or differentiation factor(s) are under the control of a constitutive promoter). Upon illumination with light (e.g., at an appropriate wavelength or wavelength range), the first and second light-homodimerizing domains dimerize, bringing the transcriptional repressor into close contact with the promoter sequence or a sequence between the promoter and the transcriptional start site. The transcriptional repressor blocks activity of the constitutive promoter and expression of the transcription factor(s) and/or differentiation factor(s) is turned off. In this scenario, those cells that are illuminated with light remain undifferentiated, whereas those cells that are not illuminated with light, or not illuminated with light at the appropriate wavelength, differentiate into a desired cell lineage.

In various aspects, the light-controllable transcriptional repressor, in the absence of light, binds to or associates with a promoter sequence (e.g., a constitutive promoter, or a sequence between a constitutive promoter and a transcriptional start site), thereby preventing or reducing expression of one or more downstream genes (e.g., one or more transcription factor and/or differentiation factor, as described herein). In the presence of light, the light-controllable transcriptional repressor does not bind to or dissociates from the promoter sequence (e.g., a constitutive promoter, or a sequence between a constitutive promoter and a transcriptional start site), thereby activating expression of one or more downstream genes (e.g., one or more transcription factor and/or differentiation factor, as described herein). In such instances, those (e.g., stem) cells that are illuminated with light differentiate into a desired cell lineage, whereas those (e.g., stem) cells that are not illuminated with light remain undifferentiated.

As shown in FIG. 4A, in one embodiment, a transcriptional repressor is fused to a first light-dissociating domain (e.g., light-dissociating domain 1). A second light-dissociating domain (e.g., light-dissociating domain 2) is fused to a DNA-binding domain. The DNA-binding domain binds to a DNA sequence within or near the promoter sequence. In the absence of light, the first and second light-dissociating domains are associated, bringing the transcriptional repressor in close contact with the promoter sequence or a sequence between the promoter and the transcriptional start site, and expression of the transcription factor(s) and/or differentiation factor(s) is turned off. Upon illumination with light (e.g., at an appropriate wavelength or wavelength range), the first and second light-dissociating domains dissociate, releasing the transcriptional repressor from the promoter sequence thereby turning on expression of the transcription factor(s) and/or differentiation factor(s). In this scenario, those cells that are illuminated with light differentiate into a desired cell lineage, whereas those cells that are not illuminated with light, or not illuminated with light at the appropriate wavelength, remain undifferentiated.

In another embodiment, as depicted in FIG. 4B, a transcriptional repressor is fused to a light-dissociating domain and a DNA-binding domain. In this scenario, the DNA-binding domain, upon homodimerizing with another DNA-binding domain, binds to a DNA sequence within or near the promoter sequence. In the absence of light, the light-dissociating domain is homodimerized with another light-dissociating domain, allowing the DNA-binding domain to homodimerize and bind the promoter sequence or a sequence between the promoter and the transcriptional start site, and bringing the transcriptional repressor into close contact with the promoter sequence or a sequence between the promoter and the transcriptional start site. The transcriptional repressor blocks activity of the constitutive promoter and expression of the transcription factor(s) and/or differentiation factor(s) is turned off. Upon illumination with light (e.g., at an appropriate wavelength or wavelength range), the light-dissociating domains dissociate, which releases the transcriptional repressor from the promoter or a sequence between the promoter and the transcriptional start site, and expression of the transcription factor(s) and/or differentiation factor(s) is turned on. In this scenario, those cells that are illuminated with light differentiate into a desired cell lineage, whereas those cells that are not illuminated with light, or not illuminated with light at the appropriate wavelength, remain undifferentiated.

Non-limiting examples of DNA binding domains that may be used with any of these embodiments include Gal4 (which binds to the Gal4 UAS DNA sequence), TetR (which binds to the TetO DNA sequence), and CymR (which binds to the CuO DNA sequence).

In some cases, the transcriptional regulator (e.g., transcriptional activator, transcriptional repressor) is fused to a light-controllable domain. In some cases, the light-controllable domain is an optogenetic switch. In some cases, the optogenetic switch is a dimerization-based optogenetic switch. In some cases, the dimerization-based optogenetic switch is a heterodimerization-based optogenetic switch, as described herein. In other cases, the dimerization-based optogenetic switch is a homodimerization-based optogenetic switch, as described herein. Any light-controllable domain may be used, including any light-controllable domain described herein. Non-limiting examples of optogenetic dimerization systems suitable for use with the methods and systems provided herein are described in Table 2.

Non-limiting examples of transcriptional activators include the transcriptional activation domain of VP16 of Herpes simplex virus (HSV), and p65. Non-limiting examples of transcriptional repressors include the KRAB (Krüppel-associated box) domain of Kox1.

In other aspects of the disclosure, control of differentiation may involve the use of a light-controllable recombinase. In such scenarios, expression of the one or more transcription factors and/or differentiation factors may be under the control of a (e.g., constitutive) promoter. A blocking sequence may be inserted between the (e.g., constitutive) promoter and the sequence encoding the one or more transcription factors and/or differentiation factors, such that expression of the one or more transcription factors and/or differentiation factors is blocked, when the blocking sequence is present. Upon removal of the blocking sequence, expression of the one or more transcription factors and/or differentiation factors is activated. Removal of the blocking sequence may be achieved by use of a recombinase (e.g., a light-controllable recombinase). In such scenarios, the blocking sequence is flanked by recombinase recognition sites, such that upon illumination with light, the light-controllable (or light-activatable) recombinase excises the blocking sequence, thereby allowing expression of the one or more transcription factors and/or differentiation factors. By fusing the recombinase to a light-controllable domain (e.g., an optogenetic switch (e.g., as described herein), the recombinase can be activated by light (e.g., at a particular wavelength or wavelength range). In such scenarios, illumination of the (e.g., stem) cells leads to differentiation of the (e.g., stem) cells to a desired cell lineage.

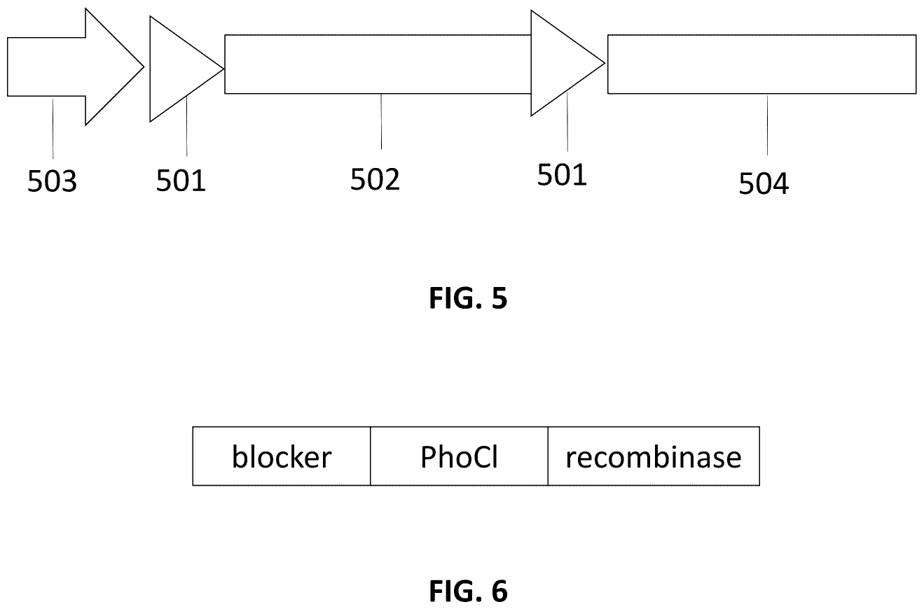

FIG. 5 depicts a non-limiting example of a nucleic acid cassette suitable for use with the disclosure herein. A promoter 503 may be operably linked to a nucleic acid sequence encoding a transcription factor 504. In some cases, a blocking sequence 502 may be located between the promoter sequence and the nucleic acid sequence encoding the transcription factor (e.g., wherein the blocking sequence is downstream of the promoter). A blocking sequence may be any nucleic acid sequence that prevents transcription of the nucleic acid sequence encoding the transcription factor, such as a nucleic acid sequence encoding one or more stop codons and/or a transcription terminator sequence. In some cases, the blocking sequence may comprise an expression cassette or multiple expression cassettes, each containing a transcription terminator sequence. In some cases, the blocking sequence may be flanked by recombinase recognition sites 501 (e.g., wherein the recombinase recognition sites are recognized by a light-controllable recombinase). In such scenarios, in the presence of an activated recombinase, the blocking sequence is excised allowing for expression of the transcription factor. In some cases, the recombinase may be activatable, e.g., by light, as described herein, thus allowing for control of recombinase activity. In such cases, the recombinase may be expressed in the (e.g., stem) cell but may be in an inactive state until the (e.g., stem) cell is exposed to light at a particular wavelength or wavelength range. Upon exposure of the (e.g., stem) cell to light at a particular wavelength or wavelength range, the recombinase may be activated, leading to excision of the blocking sequence and expression of the transcription factor (thereby leading to differentiation of the (e.g., stem) cell into a desired cell lineage).

In an alternative embodiment, rather than excision of a blocking sequence, the activatable recombinase may be used to flip nucleic acid sequences such that the nucleic acid sequences are under the control of a promoter, thereby resulting in expression of the transcription factor and/or differentiation factor.

In some cases, more than one recombinase may be used, such that, depending upon which recombinase is activated, a different transcriptional program is activated. For example, a cell may express more than one light-controllable recombinase (e.g., each fused to a different light-controllable domain). When differentiation into cell lineage A is desired, the cell may be exposed to light at a first wavelength or wavelength range, thereby activating a first recombinase fused to a first light-controllable domain, thereby resulting in expression of first transcription factors and/or differentiation factors, and differentiation of the cell into cell lineage A. When differentiation into cell lineage B is desired, the cell may be exposed to light at a second, different wavelength or wavelength range, thereby activating a second recombinase fused to a second, different light-controllable domain, thereby resulting in expression of second transcription factors and/or differentiation factors, and differentiation of the cell into cell lineage B. In a population of cells, this method may be employed to (e.g., temporally) control differentiation of cells into a specified or desired cell lineage or cell type.

In some instances, the recombinase may be activated using a light-activatable system. The light-activatable system may be as described in Table 1.

TABLE 1
Light-Activatable systems

| Purpose | Inducer | Elements |
|---|---|---|
| Optogenetically- | Light, wavelength | Split recombinase fused |
| activatable | depends on which | to halves of dimerizing |
| recombinase | optoswitches are | opto pair |
| | fused to the split | |
| | recombinase | |
| Optogenetically- | Blue light (460 nm) | LiCre |
| activatable | | |
| recombinase | | |
| Optogenetically- | Violet light (400 nm) | PhoCl, in the form |
| activatable | | SR-PhoCl-recombinase- |
| recombinase | | PhoCl-SR |

In various aspects, a combination of light-controllable domains (e.g., a first light-controllable domain and a second light-controllable domain) may be used (e.g., each of the light-controllable domains may be fused to a portion of the recombinase). In some cases, the first light-controllable domain and the second light-controlled domain can be binding partners, such that upon illumination with light at a particular wavelength or within a particular spectral range, the first and second light-controllable domains heterodimerize or hetero-oligomerize. The first and second light-controllable domains, upon illumination with light at a particular wavelength or within a particular spectral range, heterodimerize or hetero-oligomerize, thereby bringing the protein domains (or functional domains or functional portions thereof) into close contact with one another such that the recombinase(s) is/are activated.

In various aspects, the light-controllable domain comprises a Light-Oxygen-Voltage (LOV) photoreceptor domain, a LOV2 photoreceptor domain, a Cryptochrome (CRY) domain, Blue-light-using FAD (BLUF) photoreceptor domain, a Phytochrome (PHY) domain, CIB1 (cryptochrome-interacting basic-helix-loop-helix protein 1) (or a functional portion or domain thereof; e.g., CIBN (N-terminal domain of CIB1)), a PIF (phytochrome interacting factor) domain, a Dronpa domain, a UVR8 photoreceptor domain, a COP1 domain, a BphP1 domain, a QPAS-1 domain, a cobalamin-binding domain (CBD), or a combination thereof.

In some instances, a combination of light-controllable domains is used, wherein the first light-controllable domain is cryptochrome 2 (or a variant or a functional portion thereof) and the second light-controllable domain is CIB1 (or a variant or a functional portion thereof; e.g., CIBN). In some instances, a combination of light-controllable domains is used, wherein the first light-controllable domain is BphP1 (or a variant or a functional portion thereof) and the second light-controllable domain is QPAS1 (or a variant or a functional portion thereof). In some cases, the light-controllable domain (or combination of light-activatable domains) is selected from Table 2. In some cases, the light-controllable domain may have an amino acid sequence having at least about 50% sequence identity (e.g., at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or greater) to any one of the light-controllable domains described in Table 2.

TABLE 2
Non-limiting examples of light-controllable domain systems

| | Binding | Binding | Binding | |
|---|---|---|---|---|
| System | type | partner 1 | partner 2 | Light |
| Light-Oxygen- | Homodimer | VfAU1- | — | Blue light results in |
| Voltage (LOV) | | LOV | | dimerization |
| Cryptochrome | Homodimer/ | Photolyase- | — | Blue light results in |
| (CRY) | oligomer | Homologous | | dimerization |
| | | Region | | |
| | | (PHR) | | |
| | | domain | | |
| CRY-CIBN | heterodimer | PHR domain | CIBN | Blue light results in |
| | | | | dimerization |
| Blue-light- | homodimer | BLUF | — | Blue light results in |
| using FAD | | domain | | dimerization |
| (BLUF) | | | | |
| photoreceptor | | | | |
| domain | | | | |
| PhyB/PIF | heterodimer | PhyB | PIF | Red light results in |
| | | | (PIF3, | dimerization, far red |
| | | | PIF6) | light results in |
| | | | | dissociation |
| Dronpa | homodimer | Dronpa | — | Illumination with blue |
| | | | | light results in |
| | | | | monomerization; |
| | | | | illumination with UV |
| | | | | light results in |
| | | | | dimerization |
| UVR8 | homodimer | UVR8 | — | Dissociates when |
| | | | | exposed to UV-B light |
| UVR8 | heterodimer | UVR8 | COP1 | UVR8 homodimer |
| | | | | dissociates when |
| | | | | exposed to UV-B light |
| | | | | and forms heterodimer |
| | | | | with COP1 |
| BphP1/ | heterodimer | BphP1 | QPAS-1 | Near infrared light |
| QPAS1 | | | | results in dimerization; |
| | | | | red light dissociates |
| Cobalamin | oligomer | CBD | — | Homooligomerizes in |
| binding | | | | dark; green light results |
| domains | | | | in dissociation |
| (CBD) | | | | |

In another aspect of the methods described herein, the recombinase may be activated by a specific wavelength or wavelength range of light. The recombinase may be constitutively expressed in an inactive form. The recombinase may be conditionally expressed by a light-induced system in an inactive form. The recombinase may mediate irreversible excision. The recombinase may be a serine integrase, such as ϕC31, TP901, and Bxb1. The recombinase may be a tyrosine recombinase, such as Cre, VCre and Flp.

In another embodiment, dimerization and activation of the recombinase can be induced by light (e.g., optogenetic dimerization). The first half of the recombinase and the second half of the recombinase may be fused to a light-controllable domain. In various aspects, the light-controllable domain can comprise a Light-Oxygen-Voltage (LOV) photoreceptor domain, a LOV2 photoreceptor domain, a Cryptochrome (CRY) domain, Blue-light-using FAD (BLUF) photoreceptor domain, a Phytochrome (PHY) domain, CIB1 (cryptochrome-interacting basic-helix-loop-helix protein 1) (or a functional portion or domain thereof; e.g., CIBN), a PIF (phytochrome interacting factor) domain, a Dronpa domain, a UVR8 photoreceptor domain, a COP1 domain, a BphP1 domain, a QPAS-1 domain, a cobalamin-binding domain (CBD), or a combination thereof. Regulation of the dimerization and activation of the recombinase may utilize any of the light-controllable systems described in Table 2. Dimerization and activation may alternatively be induced by temperature.

In various aspects, the methods can involve exposing the cells (e.g., genetically engineered to express the fusion protein comprising the recombinase and the light-controllable domain) with light at a particular wavelength or light within a particular spectral range. The wavelength of light can be selected such that the light is capable of activating the light-controllable domain. For example, Table 2 provides non-limiting examples of light parameters for different light-controllable domain systems. The wavelength of light may be one or more of infrared, near infrared, visible light (e.g., red, green, blue), ultraviolet light, or a combination thereof. Infrared light may comprise light at a wavelength of about 780 nm to 1 mm. Near infrared light may comprise light at a wavelength of about 740 nm to about 780 nm. Red light may comprise light at a wavelength of about 620 nm to 750 nm, 600 nm to 690 nm, or about 650 nm. Green light may comprise light at a wavelength of about 577 nm to about 492 nm. Blue light may comprise light at a wavelength of 492 to about 455 nm, or about 440 nm to about 473 nm. Ultraviolet light may comprise light from about 10 nm to 400 nm, or from about 280 to 315 nm. In various aspects, the wavelength of light is from 100 nm to 1 mm.

In some embodiments, the methods and systems involve illuminating cells with light at a particular intensity or range of intensities. For example, the methods provided herein may involve illuminating cells with light at an intensity of about 2 μW/mm². In other examples, the methods provided herein may involve illuminating cells with light at an intensity of about 8 μW/mm². In some cases, the methods provided herein may involve illuminating cells with light at an intensity from about 2 μW/mm² to about 8 μW/mm². It should be understood that the level of light intensity may vary and may depend on the type of optogenetic switch used.

In some embodiments, the methods provided herein involve illuminating cells with light in an illumination pattern, such as a pulsing pattern. For example, the methods may involve illuminating cells for a period of time, turning off illumination of the cells for a period of time, and then repeating the on/off cycle for a number of times.

In another embodiment, the recombinase comprises a single chain polypeptide. The single chain polypeptide may be fused to a light-controllable domain to create a light-controllable recombinase. Exposure to light at a particular wavelength may result in activity of the recombinase. For instance, illumination of the AsLOV2-based Cre system LiCre with blue light results in activation of the recombinase.

In another embodiment, the recombinase may be fused to a PhoC1 protein or a derivative thereof. Illumination with violet light (about 400 nm) results in cleavage of the PhoC1. In some instances, a PhoC1 domain may be present in the fusion protein between a blocker domain and the recombinase domain, as depicted in FIG. 2. The blocker domain may be any domain that prevents the recombinase from functioning, such as a domain that prevents the recombinase from entering the nucleus. For instance, the blocker domain may be a steroid receptor domain which interacts with Hsp90 to prevent nuclear entry of the recombinase. Exposing the fusion protein comprising the blocker domain, the PhoC1 domain, and the recombinase to violet light may result in cleavage of the PhoC1 domain, allowing the recombinase to enter the nucleus and reach the genomic DNA, resulting in recombinase activity.

The recombinases, transcriptional regulators, transcription factors, differentiation factors, and other elements described herein can be encoded by a nucleic acid. In some embodiments, a nucleic acid comprising the recombinases, transcriptional regulators, transcription factors, differentiation factors, and other elements described herein can be an expression cassette or can be comprised within an expression cassette. As used herein, “expression cassette” means a recombinant nucleic acid construct comprising one or more nucleic acids described herein, wherein the recombinant nucleic acid construct is operably associated with at least one control sequence (e.g., a promoter).

In some cases, an engineered cell (e.g., an engineered cell described herein, for example before differentiation or after differentiation) can comprise a positive selection cassette or a negative selection cassette. For example, cells that are not of a specified or desired cell lineage or cell type (e.g., undifferentiated cells or cells differentiated to a cell lineage or type other than the specified or desired cell lineage) can be selectively killed or eliminated from a cell population by inclusion of a negative selection cassette (e.g., in a nucleic acid sequence of the cell that is not excised due to recombinase activity) or a positive selection cassette (e.g., in a nucleic acid sequence of the cell that is excised due to recombinase activity) and application of one or more factors sufficient for inducing the respective positive or negative selection cassette. In some cases, inclusion of a positive selection cassette in a nucleic acid sequence of a cell excised during recombinase activity can be used (for example, upon supplying a stimulus sufficient to activate the positive selection cassette) to remove or destroy cells in which recombinase activity has not been successful.

In certain embodiments, the nucleic acid is a component of a vector that can be used to transfer the nucleic acid into a cell. As used herein, the term “vector” refers to a nucleic acid molecule capable of transporting another nucleic acid to which it has been linked. One type of vector is a genomic integration vector, or “integration vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors”. Suitable vectors comprise plasmids, bacterial artificial chromosomes, yeast artificial chromosomes, viral vectors and the like.

In the vectors, regulatory elements such as promoters, enhancers, and polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

In some aspects, the nucleic acids that are introduced into a eukaryotic cell are operably linked to a promoter and/or to a polyA signal as known in the art. In some embodiments, the nucleic acids having a 5′ end and a 3′ end is operably linked at the 5′ end to a promoter and at the 3′ end to a polyA signal. In some aspects, the nucleic acids comprise 2A peptide sequences and/or internal ribosomal entry sites.

In some embodiments, the expression cassette includes a nucleotide sequence encoding a selectable marker, which can be used to select a transformed host cell. As used herein, “selectable marker” means a nucleotide sequence that when expressed imparts a distinct phenotype to the host cell expressing the marker and thus allows such transformed cells to be distinguished from those that do not have the marker. Such a nucleotide sequence may encode either a selectable or screenable marker, depending on whether the marker confers a trait that can be selected for by chemical means, such as by using a selective agent (e.g., an antibiotic and the like), or whether the marker is simply a trait that one can identify through observation or testing, such as by screening (e.g., fluorescence).

Advantageously, the methods herein provide for differentiating (e.g., stem) cells in suspension (rather than as, e.g., two-dimensional, adherent cell cultures). In some cases, the methods provided herein do not require the use of a feeder layer of cells. In some cases, the methods provided herein do not require the use of extracellular matrix components. In some cases, the methods involve growing or maintaining (e.g., stem) cells in a bioreactor. In some cases, the methods provided herein may involve the use of microcarriers (e.g., beads). In some cases, microcarriers may be coated with extracellular matrix components (e.g., to promote attachment of (e.g., stem) cells thereto).

In various aspects, the suspension culture has a volume of at least about 100 milliliters (mL). For example, the suspension culture may have a volume of at least about 150 mL, at least about 200 mL, at least about 250 mL, at least about 300 mL, at least about 350 mL, at least about 400 mL, at least about 450 mL, at least about 500 mL, at least about 550 mL, at least about 600 mL, at least about 650 mL, at least about 700 mL, at least about 750 mL, at least about 800 mL, at least about 850 mL, at least about 900 mL, at least about 950 mL, at least about 1000 mL, at least about 2000 mL, at least about 3000 mL, at least about 4000 mL, or at least about 5000 mL. In some cases, the suspension culture has a volume of less than about 1000 mL. In some cases, the suspension culture has a volume of greater than about 1000 mL.

Any suitable cell culture media for differentiating (e.g., stem) cells may be used. In some embodiments, the cell culture media comprises any of the following in an appropriate combination: isotonic saline, buffer, amino acids, serum or serum replacement, sugars (e.g., glucose), and other exogenously added factors. In some embodiments, the cell culture media comprises DMEM, F12, aMEM, Hepatostim™, RPMI, or combinations thereof, either in the presence or absence or serum. Suitable sera include calf serum, fetal calf serum, horse serum, or the like. In some embodiments, a serum supplement is used.

In some cases, the methods and systems provided herein may be used to differentiate (e.g., stem) cells into a desired cell lineage. In some cases, the cells used in the methods and systems provided herein are stem cells. The stem cells used in the methods and systems provided herein may be any desired stem cell. In some instances, the stem cells are pluripotent stem cells. In some instances, the stem cells are multipotent stem cells. In some cases, the stem cells are embryonic stem cells. In some cases, the stem cells are mesenchymal stem cells. In some cases, the stem cells are satellite cells or muscle stem cells. In some cases, the stem cells are fat stem cells. In certain embodiments, the stem cells described herein are mammalian stem cells. In some cases, the mammalian stem cells are selected from the group consisting of: human stem cells, bovine (cow) stem cells, ovine (sheep) stem cells, and porcine (pig) stem cells. In some cases, the stem cells are avian stem cells, such as, but not limited to, chicken stem cells. In some cases, the stem cells are fish stem cells, such as, but not limited to, tuna stem cells or salmon stem cells. In some cases, the cells may be non-stem cells (such as fibroblasts) which can be transdifferentiated into a desired cell lineage. Any cell that is capable of differentiating (e.g., any differentiatable cell) may be used in the methods and systems provided herein. Any species of cell may be used in the methods and systems provided herein, including, without limitation, human cells, bovine cells, or mouse cells.

In another aspect of the methods disclosed herein, the population of (e.g., stem) cells are deposited on a solid support, such as a microcarrier. In some cases, a population of (e.g., stem) cells can be grown on the surface of a microcarrier. In some cases, a microcarrier (or a portion thereof on which a population of cells described herein is grown) can be coated with one or more extracellular matrix components. The solid support may be biodegradable. The solid support may comprise a natural material. Natural materials include, without limitations, extracellular matrix components, silk, gelatin, and alginate. The solid support may comprise a synthetic material. The solid support may comprise any surface or scaffold to which (e.g., stem) cells can attach (e.g., hydrogel).

The solid support may be coated with one or more extracellular matrix components. For instance, the solid support may be coated with or incorporated with collagen, hyaluronic acid, fibrin, fibronectin, integrins, laminin, proteoglycans, glycosaminoglycans, gelatin, vitronectin, or any other extracellular matrix protein.

Further provided herein are systems (e.g., configured to implement the methods provided herein). In some cases, the systems comprise a population of (e.g., stem) cells in suspension culture. In some cases, at least one (e.g., stem) cell of the population of (e.g., stem) cells is genetically engineered as described herein (e.g., to contain an exogenous nucleic acid comprising a nucleic acid sequence encoding for at least one transcription factor and/or differentiation factor) that effects differentiation into a desired cell lineage. In some cases, expression of the at least one transcription factor and/or differentiation factor is controllable by light (e.g., using light-controllable transcriptional regulators, and/or light-controllable recombinases, e.g., as described herein). In some aspects, the system may further comprise one or more light source configured to illuminate at least one (e.g., stem) cell of the population of (e.g., stem) cells with light at a particular wavelength or wavelength range. In some cases, the population of (e.g., stem) cells may be grown in a suspension culture in a bioreactor. In some cases, the one or more light sources are present on the bioreactor (e.g., on a surface of a wall of the bioreactor).

In some embodiments, the one or more light source comprises one or more light-emitting diodes (LEDs). In some cases, the one or more LEDs comprises at least two different LEDs. In some cases, the at least two different LEDs emit light at different wavelengths. Additionally or alternatively, the one or more light source comprises one or more lasers. Additionally or alternatively, the one or more light source comprises an incandescent light source.

The bioreactor may be any type of culture vessel suitable for growing cells in a suspension culture. In various aspects, the bioreactor vessel comprises a total volume of at least about 50 mL, at least about 100 mL, at least about 150 mL, at least about 200 mL, at least about 250 mL, at least about 300 mL, at least about 400 mL, at least about 500 mL, at least about 600 mL, at least about 700 mL, at least about 750 mL, at least about 800 mL, at least about 900 mL, at least about 1000 mL, at least about 2000 mL, at least about 3000 mL, at least about 5000 mL, or more.

The cells used in the systems provided herein may be any desired cell, including stem cells and non-stem cells (e.g., fibroblasts). In some instances, the stem cells are pluripotent stem cells. In some instances, the stem cells are multipotent stem cells. In some cases, the stem cells are embryonic stem cells. In some cases, the stem cells are mesenchymal stem cells. In some cases, the stem cells are satellite cells or muscle stem cells. In some cases, the stem cells are fat stem cells. In certain embodiments, the stem cells described herein are mammalian stem cells. In some cases, the mammalian stem cells are selected from the group consisting of: human stem cells, bovine (cow) stem cells, ovine (sheep) stem cells, and porcine (pig) stem cells. In some cases, the stem cells are avian stem cells, such as, but not limited to, chicken stem cells. In some cases, the stem cells are fish stem cells, such as, but not limited to, tuna stem cells or salmon stem cells.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

As used herein, the term “about” a number refers to that number plus or minus 10% of that number. The term “about” a range refers to that range minus 10% of its lowest value and plus 10% of its greatest value.

In general, “sequence identity” refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the longer sequences and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (generally nucleotides or amino acids), divided by the total number of symbols in the shorter of the two sequences. The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993).

The section headings used herein are for organizational purposes only and are not to be construed as limiting the subject matter described.

### EXAMPLES

### Example 1: Recombinase-Mediated Excision to Activate Fate Cassette Expression

A population of stem cells containing the exogenous nucleic acid sequence is cultured in suspension for differentiation. A pair of recognition sites, the recognition sites being recognized by a light-controllable recombinase, is placed in the construct such that, when the corresponding recombinase is active in the cell, the muscle expression cassette (e.g., fate determining cassette) is permanently switched on. In this scheme, one recognition sequence for the recombinase is placed between the leftmost (e.g., most upstream) relevant constitutive promoter and the transcriptional start site for the gene controlled by that promoter. A fate cassette is placed at the opposite end of the construct; the fate cassette being preceded by a recognition sequence for the recombinase and a transcriptional start site, but no promoter. In this state the fate cassette should not be expressed, as there is no promoter directly upstream to recruit the transcriptional machinery due to the presence of the blocking sequence. When the cell is illuminated by light of a wavelength sufficient to activate the light-controllable recombinase and the recombinase becomes active in the cell, all of the sequence between its two recognition sites is excised, bringing the fate cassette immediately downstream of its rightmost (most downstream) recognition site under control of the leftmost (most upstream) constitutive promoter. In this case, if the recombinase is activated, the entire sequence between the promoter and the muscle expression cassette is excised, the muscle expression cassette is expressed.

### Example 2: Light-Induced Differentiation of Fibroblasts into Adipocyte-Like Cells in Suspension Culture

This example demonstrates light-induced differentiation of fibroblasts into adipocyte-like cells in suspension culture. In this example, a photoactivatable Cre recombinase was used to excise a blocking sequence and drive expression of the adipocyte transcription factors, peroxisome proliferator-activated receptor gamma (PPARg) and CCAAT/enhancer-binding protein alpha (CEBPa).

### Plasmids/Constructs

The plasmids created for this study were designed in Geneious (Biomatters). The different components of the plasmids were either obtained by restriction digest, PCR, or via DNA synthesis and assembled via Gibson cloning. Table 3 describes the plasmids used in these studies. The pCMV-PPARg, the Cre reporter plasmid, and the SV40 lentivirus were obtained from commercial sources.

TABLE 3
Plasmids/viral vectors

| Name | Type | Construct Details |
|---|---|---|
| 703 | Plasmid | P_{Eflα}-loxP-3xSV40pA-loxP-PPARG-IRES-mScarletI-rBGpA-P_{CMV}- |
| | | Neo-BGHpA |
| 704 | Plasmid | P_{Eflα}-loxP-3xSV40pA-loxP-PPARG-T2A-CEBPA-IRES-mScarletI- |
| | | rBGpA-P_{CMV}-Neo-BGHpA |
| 361 | Plasmid | P_{CMV}-NLS-Cre-BGHpA |
| 709 | Plasmid | P_{Eflα}-NLS-LiCre(AsLOV2-Cre)-IRES-mTagBFP2-bGHpA |
| P_{CMV}- | Lentiviral | P_{CMV}-PPARG-WPRE-P_{mPGK}-Puro |
| PPARg | vector | |
| Cre reporter | Plasmid | pMSCV-loxp-dsRed-loxp-eGFP-PPGK-Puro-WPRE |
| plasmid | | (lifescienceMarket, cat. no. PVT11052) |
| SV40 lenti | Lentiviral | P_{CMV}-SV40 large T-antigen-PRSVhygro (Gentarget Inc, LVP016- |
| virus | vector | Hygro, San Diego, CA) |

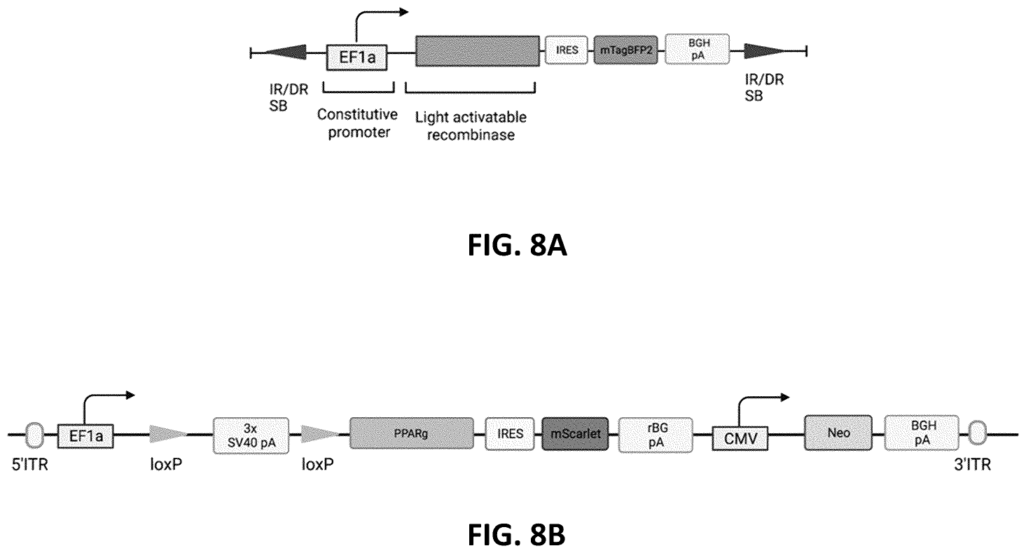

The plasmid 703 (schematic shown in FIG. 8B) comprises a nucleic acid sequence encoding the bovine transcription factor PPARg (UniProt: O18971-1) and the fluorescent protein mScarletI (GenBank: APD76536.1) under control of the constitutive human Ef1α promoter (P_{Ef1α}). The coding sequences of PPARg and mScarletI are connected by an IRES (internal ribosome entry site) sequence to allow co-expression of both genes. PPARg-IRES-mScarletI is connected to the promoter via a sequence comprising three SV40 polyadenylation sequences (SV40 pA) flanked by loxP sites. Recombination of the two loxP sites via Cre recombinase excises the sequence comprising the three SV40 polyadenylation sequences and turns on expression of PPARg and mScarletI. Transcription from the Ef1α promoter is terminated via the rabbit β-globin polyadenylation signal (rBGpA). The plasmid further comprises an expression cassette for the neomycin resistance gene (Neo, GenBank: WP_004614937.1) comprising the constitutive human cytomegalovirus immediate early promoter (P_{CMV}) and the bovine growth hormone polyadenylation signal (bGHpA), which allows for selection of cells with genomic integration of the described plasmid elements. The entire nucleotide sequence comprising the described elements is flanked by the 5′ and 3′ ITR sequences of the PiggyBac transposon.

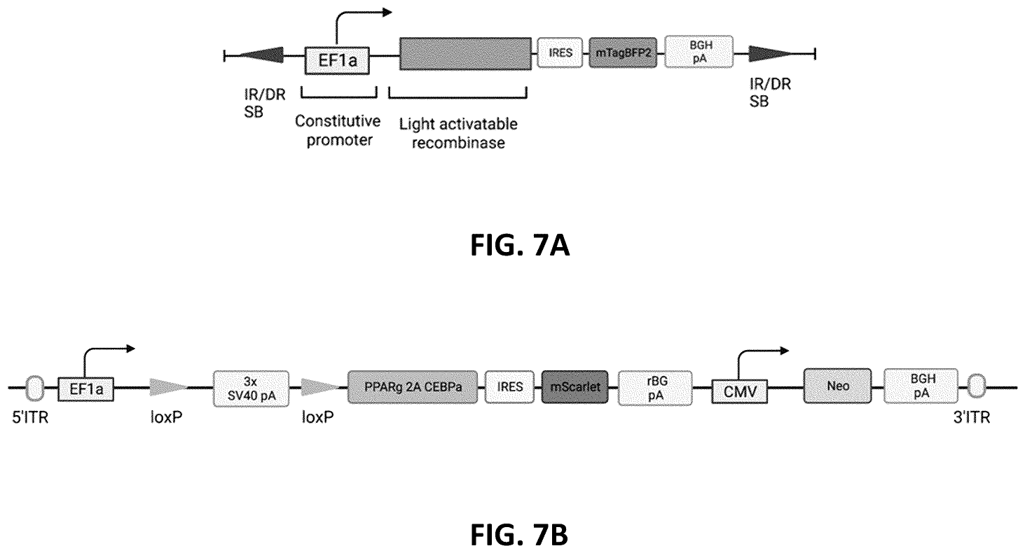

The plasmid 704 (schematic shown in FIG. 7B) comprises a nucleic acid sequence encoding the bovine transcription factors PPARg (UniProt: O18971-1) and CEBPa (GenBank: NP_789741.2) and the fluorescent protein mScarletI (GenBank: APD76536.1) under control of the constitutive human Ef1α promoter (P_{Ef1α}). The coding sequences of PPARg and CEBPa are connected via a T2A self-cleaving peptide and PPARg-T2A-CEBPa is connected to mScarletI by an IRES (internal ribosome entry site) sequence to allow co-expression of the three genes. PPARg-T2A-CEBPa-IRES-mScarletI is connected to the promoter via a sequence comprising three SV40 polyadenylation sequences (SV40 pA) flanked by loxP sites. Recombination of the two loxP sites via Cre recombinase excises the sequence comprising the three SV40 polyadenylation sequences and turns on expression of PPARg, CEBPa, and mScarletI. Transcription from the Ef1α promoter is terminated via the rabbit β-globin polyadenylation signal (rBGpA). The plasmid further comprises an expression cassette for the neomycin resistance gene (Neo, GenBank: WP_004614937.1) comprising the constitutive human cytomegalovirus immediate early promoter (P_{CMV}) and the bovine growth hormone polyadenylation signal (bGHpA), which allows for selection of cells with genomic integration of the described plasmid elements. The entire nucleotide sequence comprising the described elements is flanked by the 5′ and 3′ ITR sequences of the PiggyBac transposon.

The plasmid 361 comprises an expression cassette comprising a constitutive human cytomegalovirus immediate early promoter (P_{CMV}), a coding sequence for the Cre recombinase (UniProt: P06956, residues 2-343) linked to a nuclear localization signal (UniProt: Q2HJ27, residues 320-328), and the bovine growth hormone polyadenylation signal (bGHpA).

The plasmid 709 (schematic depicted in FIG. 7A and FIG. 8A) comprises an expression cassette comprising the constitutive bovine Ef1α promoter (P_{Ef1α}), a coding sequence for a light-inducible Cre recombinase and the fluorescent protein mTagBFP2 (GenBank: HCE4034942.1), and the bovine growth hormone polyadenylation signal (bGHpA). The coding sequences of the light-inducible Cre recombinase and of mTagBFP2 are connected by an IRES (internal ribosome entry site) sequence to allow co-expression of both genes. The light-inducible Cre recombinase comprises three nuclear localization signals (UniProt: Q2HJ27, residues 320-328) fused to the N-terminus of an N-terminally truncated version of LiCre (as described in Duplus-Bottin et al., “A single-chain and fast-responding light-inducible Cre recombinase as a novel optogenetic switch”, eLife 10:e61268 (2021)) which comprises AsLOV2 (Genbank: 4WF0_A, residues 11-148) and Cre (UniProt: P06956, residues 20-339). The entire nucleotide sequence comprising the described elements is flanked by the 5′ and 3′ IR/DR sequences of the Sleeping Beauty transposon.

The P_{CMV}-PPARg lentiviral vector comprises a nucleic acid sequence encoding the bovine transcription factor PPARg (UniProt: O18971-1) under control of the constitutive human cytomegalovirus immediate early promoter (P_{CMV}). The lentiviral vector further comprises a Woodchuck hepatitis virus posttranscriptional regulatory element (WPRE) and a nucleic acid sequence encoding a puromycin resistance gene under control of the constitutive mouse phosphoglycerate kinase 1 promoter (P_{mPGK}).

### Cell Lines

Isolation of Fibroblasts from Wagyu

Adipose stem cells were extracted from fresh adipose tissue samples of adult Wagyu bull. Briefly, adipose tissue was minced with a sterile scalpel, added to 25 ml digestion solution (2 mg/ml Collagenase II (Sigma, C2-BIOC), 1% penicillin/streptomycin/amphotericin B (Lonza, 17-745E), 10 μM ROCK inhibitor Y-27632 (Tocris, 1 254)), leading to a total volume of 40 ml in a 50 ml centrifuge tube, and incubated at 37° C. for 60 minutes with inversion every 2 minutes. The tube was centrifuged at 300×g for 5 minutes, resulting in a pellet at the bottom and a plug of fat at the top, which was discarded along with the supernatant. The pellet was washed with 25 ml growth medium (DMEM (Sigma, SLM-021) with 10% fetal bovine serum (Avantor, 89510-186) and 1% penicillin/streptomycin/amphotericin B, then resuspended in 10 ml growth medium and transferred to a T75 tissue culture flask. Cells were incubated undisturbed at 37° C. and 5% CO₂ for 24 hours, and thereafter were expanded for multiple passages with changes of growth medium every 2 days.

### Creation of SV40 Background Cell Line (SV40 Cells)

Cells stably expressing SV40-LgT (Large T antigen) were created as they confer a doubling time advantage and immortalizes the cells. P3 Wagyu fibroblast cell line was cultured in DMEM supplemented with 10% FBS and 1% penicillin/streptomycin/amphotericin B, and maintained under standard conditions. The cells were transduced with lentivirus (carrying LgT under CMV promoter, Gentarget Inc LVP016-Hygro San Diego, CA) in a 24-well plate, with 20 μl of virus added to each well, and then incubated for 24 hours. Post incubation, the medium was replaced with fresh culture medium. Approximately 48 hours later, cells were subjected to antibiotic selection (Hygromycin, InvivoGen, San Diego, CA) for a week, promoting survival of only stably transduced cells with the antibiotic resistance gene. These cells were then expanded and, upon reaching adequate numbers, some were cryopreserved for future use, involving resuspension in a freezing medium, controlled-rate freezing, and storage in liquid nitrogen.

### Integration of Plasmids Using PiggyBac/Sleeping Beauty and Selection Using Geneticin/Cell Sorting

500,000 SV40 cells were seeded in a T75 flask in growth medium the day before transfection. On the day of transfection, each transposon plasmid was co-transfected with a transposase plasmid (hyPB for PiggyBac and hySB100X for Sleeping Beauty) using Lipofectamine™ 3000 Transfection Reagent (ThermoFisher) according to the manufacturer's instructions. Each flask contained 9 μg of transposon plasmid, 0.9 μg of the corresponding transposase plasmid, 22 μL P3000, and 27 μL Lipofectamine™ 3000. Transfection without transposase was also performed to monitor a decrease in transient transfection signal before cell sorting. Medium was replaced 48 hours after transfection. For plasmid 703 and plasmid 704 transfected cells, 4 mg/ml of geneticin was included in the growth medium for selection. A control flask containing the same number of cells but not engineered was selected with geneticin simultaneously. All the cells in the control flask were killed on day 6 which indicated the surviving cells in the experimental flask were engineered. Engineered cells were expanded in growth medium without geneticin for experiments.

### Sorting Populations of Cells Stably Expressing the Plasmid

Cells were detached and dissociated into single cell suspension using TrypLE by incubation at 37° C. for 5 minutes. After centrifugation, cells were resuspended in PBS and filtered through 100 μm cell strainer. Cell sorting of PiggyBac and Sleeping Beauty transposon integrated cell populations were performed using SONY cell sorter SH800S according to the manufacturer's instruction. For sorting plasmid 709 in 703 and 704-transfected cells, gating on mScarlet negative BFP low and mScarlet negative BFP high populations was performed to purify Cre recombinase expressing cells with an intact loxP-3×STOP-loxP sequence upstream of the transcription factors. All the sorted cell populations were expanded in the growth medium.

### Bioreactor Preparation

100 ml spinner flasks with impellers (Bellco) were coated with Sigmacote (Sigma Aldrich), excess Sigmacote was removed and the flasks were dried by removing the side caps and lid, inverting the flasks and placing them in a fume hood overnight. The following day, the flasks were rinsed with 100 ml fresh MilliQ water and dried. Dry reactors were assembled and autoclaved for 30 minutes on a fast/dry cycle.

### Microcarrier Preparation

50 ml falcon tubes were coated with Sigmacote, dried, washed with fresh MilliQ water and dried upside down before use. 0.2 g Cytodex 3 microcarriers were used per 100 ml flask. The microcarriers for each spinner flask were placed in the coated 50 ml falcon tubes, and hydrated by adding 45 mL Mg/Ca²⁺ free PBS to each falcon tube. The falcon tubes were inverted a few times until the microcarriers were fully suspended, then left for at least 3 hours at room temperature. The supernatant was removed and the microcarriers were washed again with Mg/Ca²⁺ free PBS. After removing the supernatant Mg/Ca²⁺ free PBS was added up to 40 ml mark and the microcarriers were sterilized by autoclaving for 15 minutes on the wet cycle. The rehydrated Cytodex 3 could be stored at 4° C. or used straight away. To use the microcarriers, 25 ml PBS supernatant was removed and 25 ml of growth media containing DMEM/F12 high glucose, 10% FBS, 1% penicillin/streptomycin/amphotericin B. The microcarriers were left to settle and the media wash was repeated 2 times. For a 100 ml vessel, the volume was brought up to 20 mL.

### Suspension Culture

The microcarrier solution (20 ml) was inverted and poured into each spinner flask, the falcon tubes were rinsed with 20 ml growth media containing DMEM/F12 high glucose+10% FBS+1% penicillin/streptomycin/amphotericin B, which was added to the flasks. Spinner flasks were then placed onto a spinner plate and were spun at 30 rpm for at least 2 hours before adding cells. Cells were washed, trypsinized and counted. 2-3 million cells were added to each reactor and the volume was brought up to 100 ml using growth media. The spinner flasks were closed and swirled to gently mix the cells and microcarriers. The flasks were then placed in the incubator and spun for 1 minute at 30 rpm every hour for 3 hours, before being left to attach to the microcarriers overnight, without spinning.

The following day the spinner flasks were placed on the spinner at 30 rpm continuously. For each experiment, one flask was exposed to light while another flask was not exposed to any light as a control. The blue light (peak intensity at approx. 465 nm) used to illuminate the flask was administered in 20 second pulses every 80 seconds with an approximate power density of 8 μW/mm² at the reactor wall. All the flasks were covered by light-proof sheath and base together with aluminum foil to prevent leakage of light into the dark control flasks. Illuminated flasks were exposed to the illumination pattern for 48 hours. After 2 days of suspension culture, growth media was exchanged for StemPro adipogenesis differentiation kit media (Gibco) supplemented with 1% penicillin/streptomycin/amphotericin B. Thereafter, 70% StemPro media was exchanged on day 3. To do this, spinner flasks were taken off the spinner plate and microcarriers were allowed to settle for 5 minutes, before the 70 ml warmed StemPro media was exchanged. 1 mL of microcarrier/media was sampled per well for imaging, which was performed in triplicate for each sample. Where shown, 2 mLs of microcarrier/media were sampled for RNA in duplicate. Cultures were sampled after 6 days in StemPro media

### Staining

To stain with BODIPY 493/503, wells were topped up with PBS, most of the supernatant was removed without exposing the cells to air, 1 mL/well of PBS (24-well plate) containing 260 ng/ml of BODIPY 493/503 was added gently to the well. The plate was incubated at 37° C., 5% CO₂ and a humidity of approximately 95% in a cell culture incubator for 30 minutes. 1 mL of supernatant was gently removed from each well, and 1 mL of PBS was gently added back to each well for imaging.

To stain with Hoechst, 1 μg/ml of Hoechst in PBS was added to the sample for 30 minutes together with the BODIPY 493/503 stain.

### Imaging

All images, 2D and 3D, were acquired using a Leica dmi8 THUNDER microscope. More specifically, the 20× zoom with 0.8 aperture objective was used. To enhance image quality and reduce noise, the THUNDER's Instant Computational Clearing (ICC) method was applied to every image upon acquisition. The intensities, exposure times, and excitation/emission wavelengths of the Leica filter cubes for all fluorescent stains are listed below:

### Hoechst:

Intensity: 31%
Exposure time: 100 ms
Excitation wavelength: 390 nm
Emission wavelength: 460 nm

### BODIPY 493/503:

Intensity: 35%
Exposure time: 300 ms
Excitation wavelength: 510 nm
Emission wavelength: 535 nm

### 2D Imaging

For all 2D assays, cells were imaged in 96-well glass-bottom plates. On the 96-well plates, 6×6 tile scans were taken. For all tile scans, the Leica AFC laser autofocus was used to ensure that images were in focus across wells.

### 3D Imaging

For the assay comparing MBX, SV40, and SV40-PPARg cells in Growth and Stempro media (see below), images were acquired on a 6-well glass bottom plate. All other 3D images were taken in 24-well glass-bottom plates. For all suspension experiments on both types of plates, 2 Z-stack images were manually taken at different locations within each well to ensure microcarriers were imaged. For each z-stack, locations were picked that were dense with microcarriers, to ensure similar samples were taken across different wells and experiments.

### Image Analysis

For all image analysis, after post-processing on the Leica as described below, images were exported from the Leica .lif files into individual .tif files using python. Additionally, all plotting was performed using python. Across all suspension experiments, a normalized BODIPY spot area occupied per image was chosen as the key metric, as BODIPY spots often conglomerated together. As such, area occupied was seen as a reliable measure of BODIPY stain and thus lipid droplet accumulation. Across all analyses, any segmented BODIPY objects that had a diameter less than or equal to 2 pixels in size were filtered out.

### 2D Image Analysis

First, tile scans were stitched together into a single image using the Leica software's Mosaic Merge function. Exported images were loaded into a CellProfiler pipeline. To segment BODIPY spots in 2D, CellProfiler's RobustBackground segmentation algorithm was used.

### 3D Image Analysis

Z-stack images were processed using the Leica's Max Projection function. This means that for every pixel in every image, the maximum intensity value for each channel across all z-planes was represented, resulting in 2D images. These exported images were loaded into CellProfiler as well. To segment BODIPY stain on microcarriers, CellProfiler's Otsu segmentation algorithm was found to be best. As such, all max projected images were segmented using the Otsu algorithm. CellProfiler's implementation of Otsu was found to perform better than the Leica implementation for segmenting BODIPY stain on microcarriers. For data normalization, microcarrier counts were performed manually for each image.

qPCR

RNA was isolated from collected cell samples utilizing Zymo Research's Direct-zol RNA Miniprep kit and TRI Reagent, following strict RNase-free practices. Subsequently, the extracted RNA was quantified and transcribed into cDNA via the Applied Biosystems™ High-Capacity RNA-to-cDNA™ kit. For qPCR, the cDNA was amplified using Applied Biosystems™ PowerUp™ SYBR™ Green Master Mix, with the inclusion of appropriate primers. The prepared reaction mix was loaded into a Quantstudio 7 Pro instrument. Amplification conditions were set as follows: UDG activation at 50° C. for 2 minutes, Dual-Lock DNA Polymerase activation at 95° C. for 2 minutes, and 40 cycles of denaturation at 95° C. for 15 seconds and annealing/extension at 60° C. for 1 minute. The program was run in standard mode as per the manufacturer's instructions. Fold change light vs dark was calculated via 2\hat{ }−delta delta Ct.

Fibroblasts Constitutively Expressing PPARg Grown in Adipogenesis-Inducing Media Differentiate into Adipocyte-Like Cells

This example demonstrates that fibroblasts constitutively expressing the adipogenesis master regulator, PPARg, differentiate to fat cells in both two-dimensional culture and suspension culture, when grown in adipogenic-inducing media, but not when in adipogenesis-inducing media without constitutive PPARg expression.

In this example, wild-type bovine fibroblasts, as well as fibroblasts which had been transduced with SV40 large T antigen (SV40-LgT) (as described above) were used. The SV40-LgT gene expression confers a proliferation advantage, reducing doubling time of the fibroblasts. 300,000 wild type (WT) or SV40-expressing fibroblast cells were seeded into a 100 mm dish in growth medium containing DMEM/F12+10% FBS+1% penicillin/streptomycin/amphotericin B the day before transduction. The next day, growth medium was replaced before adding CMV-PPARg lentiviral vector (Vector Builder) at MOI 10 and polybrene at 8 μg/ml for transduction. Cells were incubated with lentiviral vectors for 48 hours before selection with puromycin at 1.5 μg/ml in growth medium. A control plate containing the same number of cells but not transduced with the viral vector was selected with puromycin simultaneously. All the cells in the control plate were killed on day 6 which indicates the surviving cells in the transduced plate were transduced with CMV-PPARg viral vectors. Transduced cells were expanded in a growth medium containing DMEM/F12+10% FBS+1% penicillin/streptomycin/amphotericin B without puromycin for experiments.

To demonstrate differentiation of fibroblasts to fat cells in two-dimensional culture, WT cells, WT cells transduced with CMV-PPARg, SV40 cells and SV40 cells transduced with PPARg were first seeded at 3000 cells/cm² in growth media containing DMEM/F12+10% FBS+1% penicillin/streptomycin/amphotericin B for 24 hours to enable cell attachment. The next day, media was exchanged for StemPro adipogenesis differentiation medium. Medium was replaced after 3 days by gently removing supernatant without disturbing the oil droplets in the cells. Cells were cultured for 6 days in adipogenesis media. Lipid staining was conducted by staining with BODIPY 493/503 as described above. Imaging and image analysis was performed as described above.

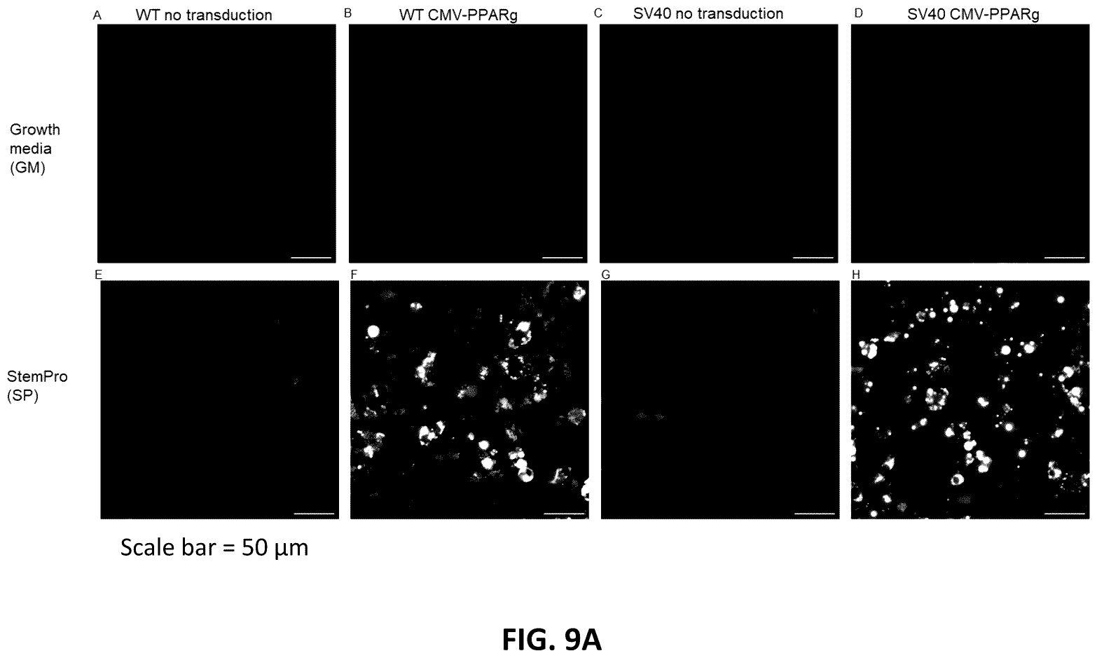

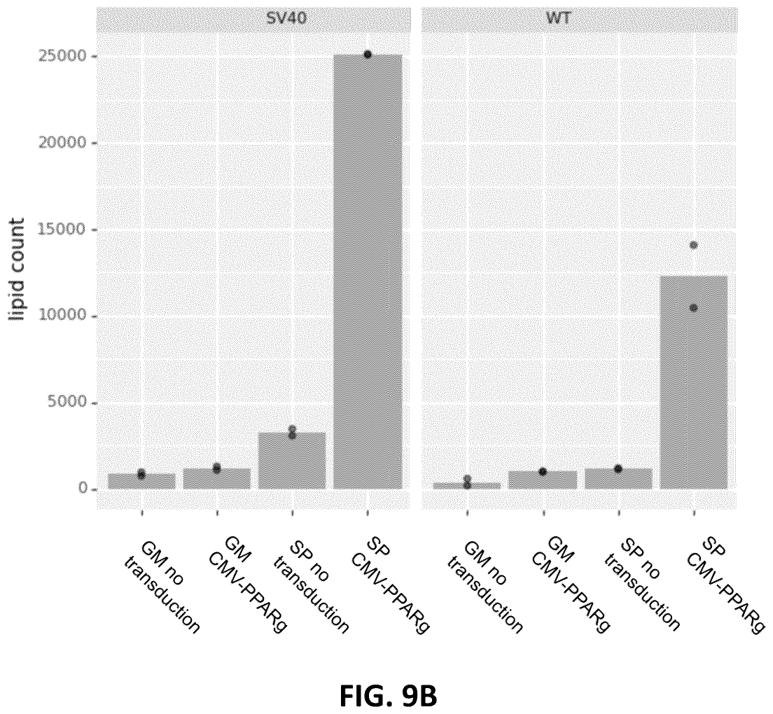

As shown in FIGS. 9A and 9B, constitutive expression of PPARg in both the WT cells (FIG. 9A, panel F) and the SV40-expressing cells (FIG. 9A, panel H) led to enhanced lipid droplet formation (stained using BODIPY 493/503) when in the presence of StemPro media, an adipogenesis-inducing media, but not when in the presence of growth media (FIG. 9A, panels B and D). Adipogenic media alone did not lead to enhanced lipid droplet formation (FIG. 9A, panels E and G), demonstrating PPARg expression in fibroblasts grown in adipogenic media is necessary for lipid droplet accumulation and thus fibroblast differentiation towards adipocyte-like cells in 2-dimensional cell culture. FIG. 9B depicts a quantification of FIG. 9A. As shown in FIG. 9B, when cells constitutively expressing PPARg are grown in growth media, lipid droplet accumulation is minimal. Similarly, when cells are grown in adipogenic media without PPARg expression, there is also minimal lipid droplet accumulation. In contrast, when cells constitutively expressing PPARg are grown in adipogenic media, there is robust lipid droplet accumulation.

These results were further confirmed in three-dimensional suspension culture. Spinner flasks were prepared as described above. Fibroblasts containing SV40 cells, with and without constitutive expression of PPARg (as described above), as well as 3T3-L1 MBX cells were cultured in growth media containing DMEM/F12+10% FBS+1% penicillin/streptomycin/amphotericin B. The cells were seeded onto Cytodex 3 microcarriers as described above, and the cells were cultured in suspension as described above, with the exception that in some spinner flasks, growth media was switched to StemPro one day after inoculation. Growth media and StemPro were exchanged on day 3 and the samples were harvested on day 6. Cells/microcarriers were stained according to the BODIPY 493/503 stain described above. 3T3-L1 MBX cells were used as a positive control. 3T3-L1 MBX cells are a fibroblast cell line that was derived to ensure close to 100% differentiation towards adipocyte-like cells when grown in adipogenic media.

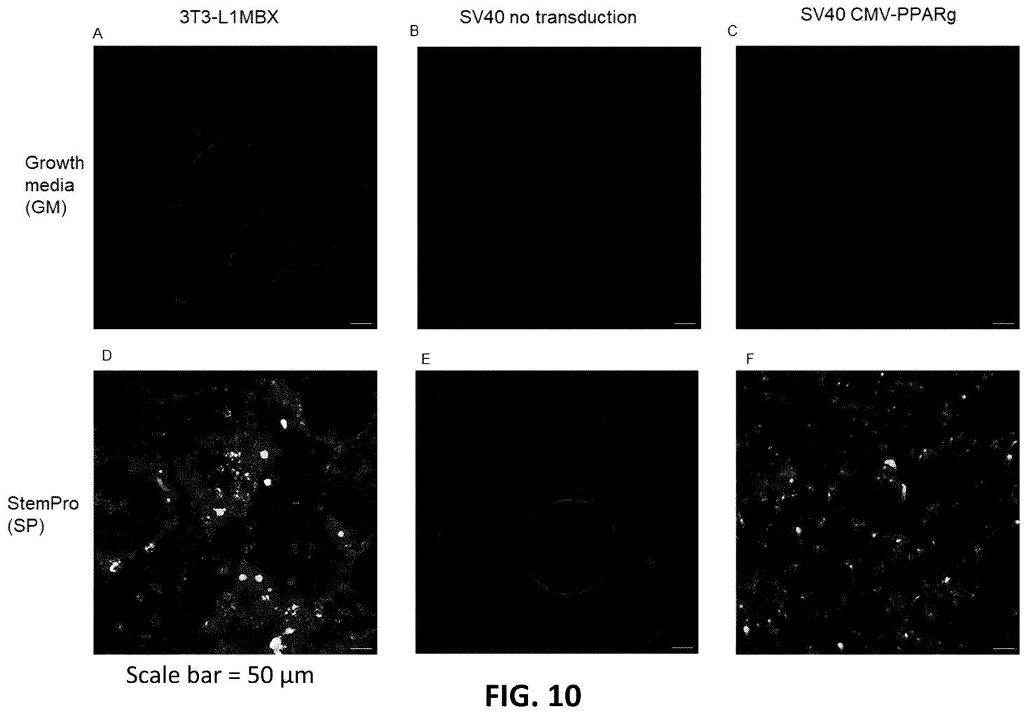

As depicted in FIG. 10, the 3T3-L1 MBX cells as well as the SV40-CMV PPARg cells demonstrate lipid droplet accumulation when grown in adipogenic media (FIG. 10, panels D and F, respectively), but not when grown in growth media (FIG. 10, panels A and C, respectively). Non-transduced cells do not express adipocyte-like lipid droplets when grown in growth media or adipogenic media (FIG. 10, panels B and E, respectively), demonstrating that expression of PPARg plus adipogenic media is necessary for differentiation of fibroblasts towards fat. This example confirms that fibroblasts can be differentiated towards fat in suspension culture.

### Validation of a Light-Activatable Recombinase Construct

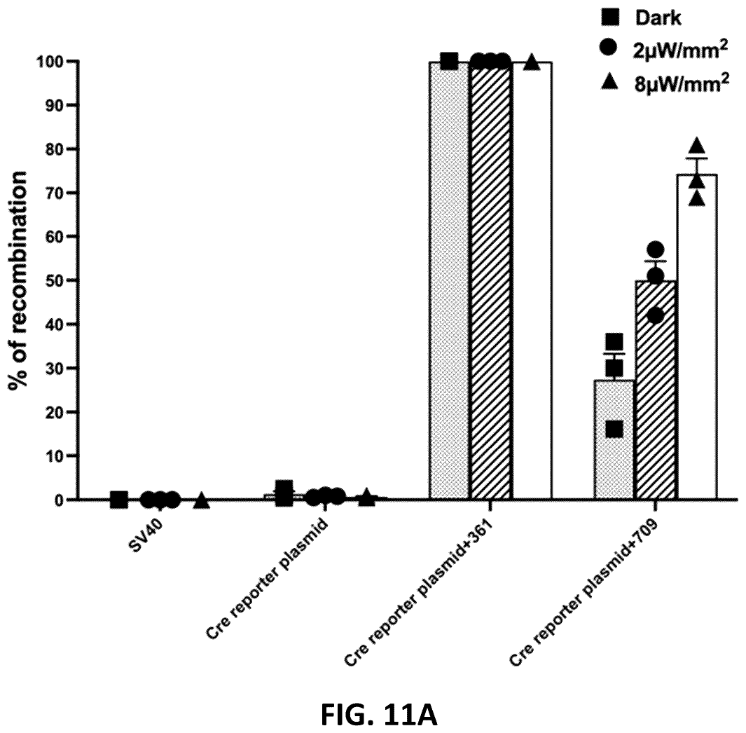

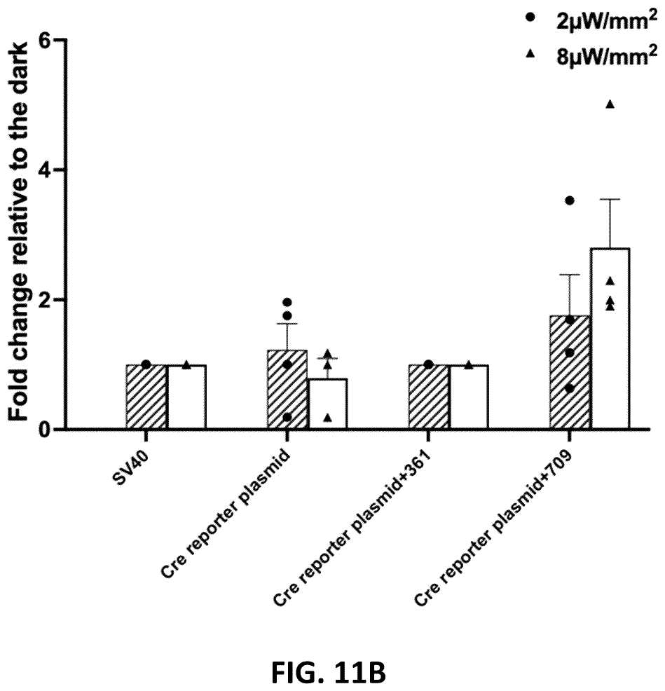

In this example, a plasmid encoding a light-activatable recombinase was tested and validated for use in subsequent experiments. SV40 cells were seeded at a density of 6000 cells/well to a 96-well glass-like polymer bottom plate in growth medium the day before transfection. On the day of transfection, plasmid 709 (light-activatable Cre recombinase) or plasmid 361 (wild-type Cre recombinase) was co-transfected with a Cre recombinase reporter plasmid (pMSCV-loxp-dsRed-loxp-eGFP-Puro-WPRE, lifescienceMarket, cat. no. PVT11052) using Lipofectamine™ 3000 Transfection Reagent (ThermoFisher) according to the manufacturer's instructions. Each well contained 30 ng of Cre plasmid, 30 ng of the Cre reporter plasmid, 0.12 μL P3000, and 0.2 μL Lipofectamine™ 3000. The Cre recombinase reporter plasmid expresses the fluorescent protein dsRed until Cre recombinase activity excises the coding sequence for dsRed located between the two loxP sites and turns on expression of the fluorescent protein eGFP. Cells were handled under red or green light since room light activates the recombinase and permanently alters the DNA. Medium in each well was replaced 24 hours after transfection before starting illumination in selected wells (2 μW/mm² or 8 μW/mm² of pulsed blue light (465 nm) (20 seconds ON, 60 seconds OFF). Cells were harvested for flow cytometry (Attune CytPix, Thermo Fisher Scientific) analysis 48 hours after start of illumination. The recombination efficiency was calculated as follows: % of recombination=% of EGFP expressing cells/% of EGFP and % mCherry expressing cells, and plotted as shown in FIG. 11A; or the recombination fold change in light vs dark was calculated and plotted, as depicted in FIG. 11B.

As shown in FIGS. 11A and 11B, there was minimal leaky recombination when no Cre recombinase was present. Constitutive expression of Cre (CMV-Cre) resulted in ˜100% efficiency of recombination validating the reporter plasmid. The light-activatable recombinase (plasmid 709) was validated and showed a light power-dependent increase in recombination, with a peak recombination at 8 μW/mm², of the two light intensities tested.

### Differentiation of Fibroblasts to Fat Cells Using Constitutively Expressed Recombinase to Excise a Blocking Sequence and Drive Expression of Adipogenic Transcription Factors.

In this example, fibroblasts were differentiated to fat cells in two-dimensional cell culture using a constitutive Cre recombinase. This example validates the plasmids used to differentiate fibroblasts to fat cells.

6000 cells/well of cells stably expressing plasmid 703 or plasmid 704 (see Table 3) were seeded into a 96-well glass bottom polymer plate in growth media (containing DMEM/F12 high glucose, 10% FBS, 1% penicillin/streptomycin/amphotericin B) the day before transfection. On the day of transfection, transfection of CMV-Cre plasmid (plasmid 361) was performed using Lipofectamine™ 3000 Transfection Reagent (ThermoFisher) according to the manufacturer's instructions. Non-transfected controls were exposed to the transfection reagents alone. Each transfected well contained 60 ng of CMV-Cre, 0.12 μL P3000, and 0.2 μL Lipofectamine™ 3000. 48 hours after transfection, media in some wells was changed to StemPro adipogenic differentiation medium for 6 days. Growth media and adipogenic media were replaced after 3 days. BODIPY 493/503 staining of lipid droplets was performed on day 6 according to the procedure outlined above.

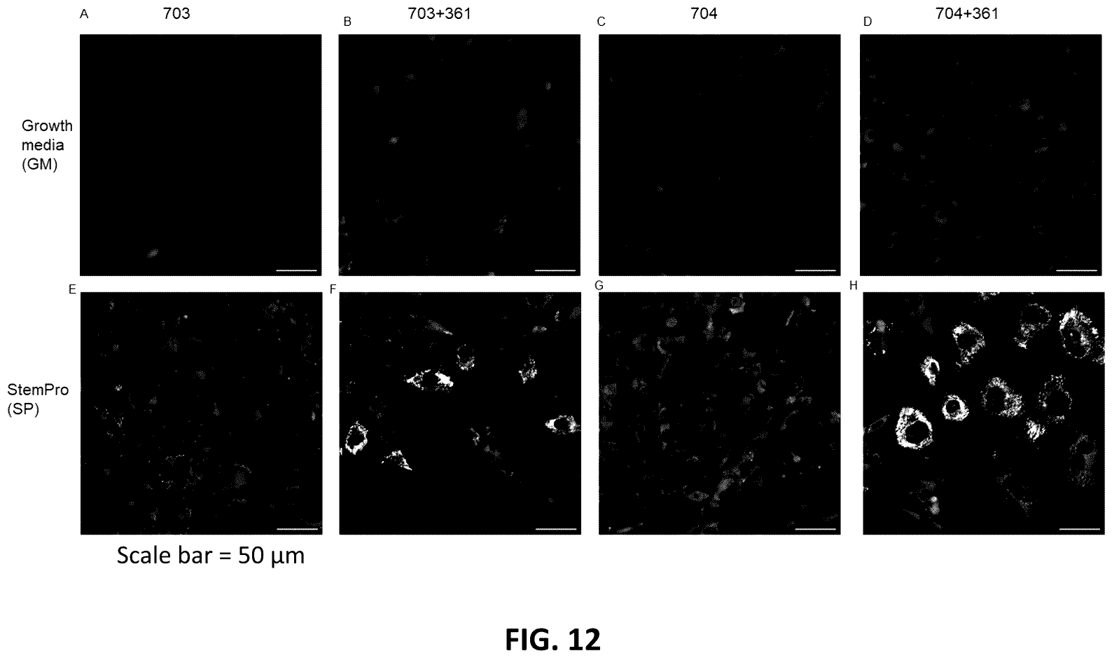

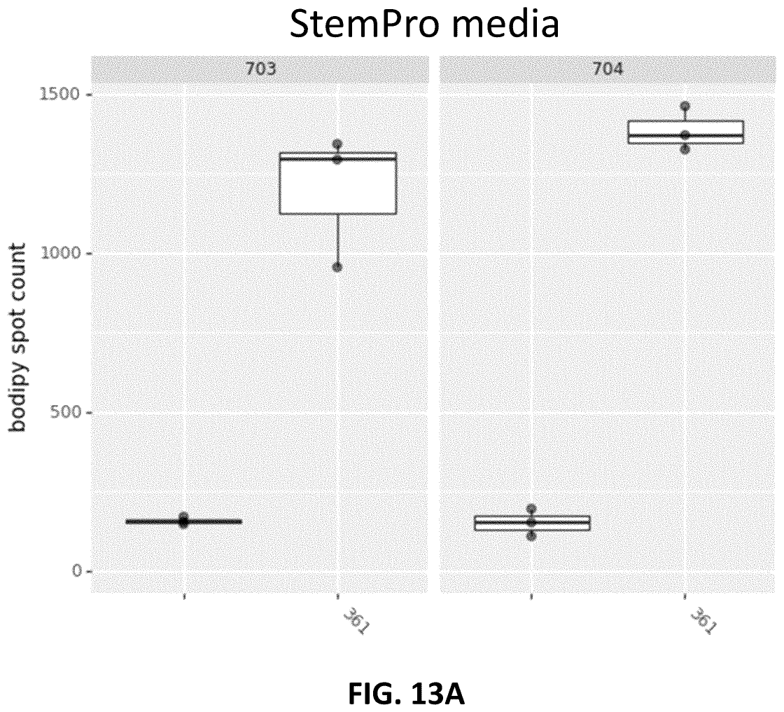

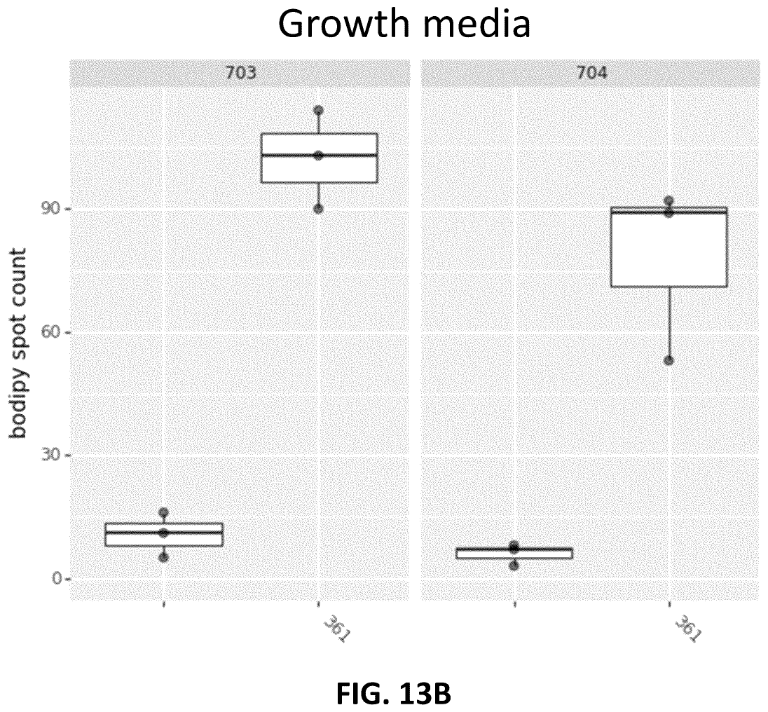

As shown in FIG. 12, more lipid droplets were stained in cells which were transfected with plasmids 703 and 361 (FIG. 12, panels B and F) or with plasmids 704 and 361 (FIG. 12, panels D and H), than in cells transfected with plasmids 703 (FIG. 12, panels A and E) or 704 (FIG. 12, panels C and G) alone in respective StemPro or growth media experiments. This demonstrated that constitutively expressed Cre recombinase was able to excise the blocking sequence of plasmids 703 and 704 such that expression of transcription factors (PPARg for plasmid 703; or PPARg and CEBPa for plasmid 704) was turned on and differentiation to adipocyte-like cells occurred. Expression of PPARg alone and PPARg+CEBPa led to substantially more lipid droplet accumulation when in StemPro adipogenic media (FIG. 12, panels F and H, respectively) than in growth media (FIG. 12, panels B and D), once again highlighting that both transcription factors and adipogenic media were necessary for differentiating fibroblasts towards fat. FIG. 13A and FIG. 13B depict quantification of the results shown in FIG. 12.

Differentiation of Fibroblasts into Fat Cells Using Light-Activatable Recombinase to Excise a Blocking Sequence and Drive Expression of Adipogenic Transcription Factors

In this example, fibroblasts were differentiated to fat cells in both two-dimensional cell culture and three-dimensional suspension culture using a light-activatable Cre recombinase.

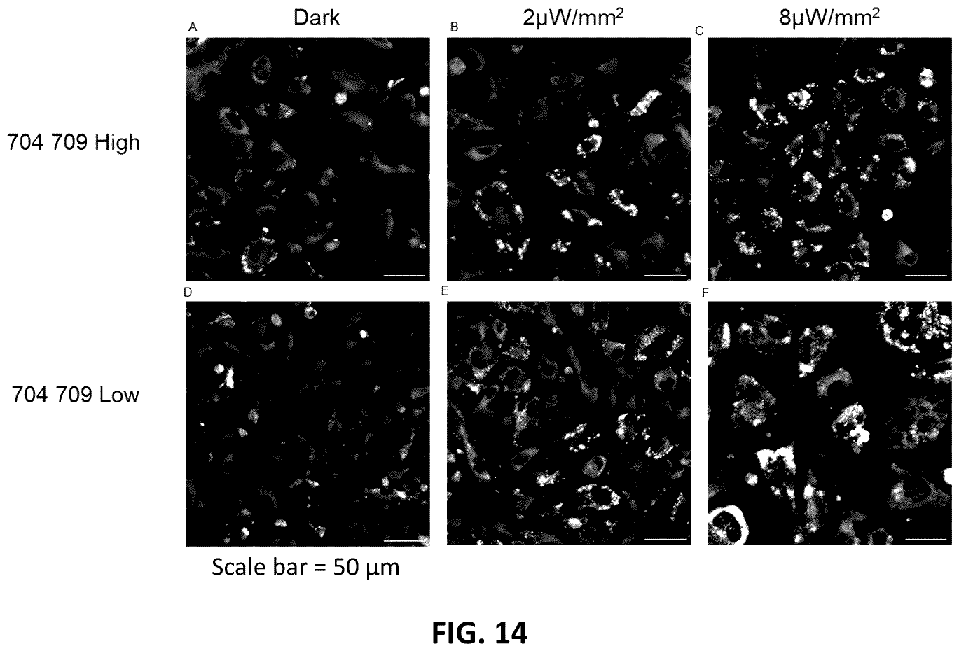

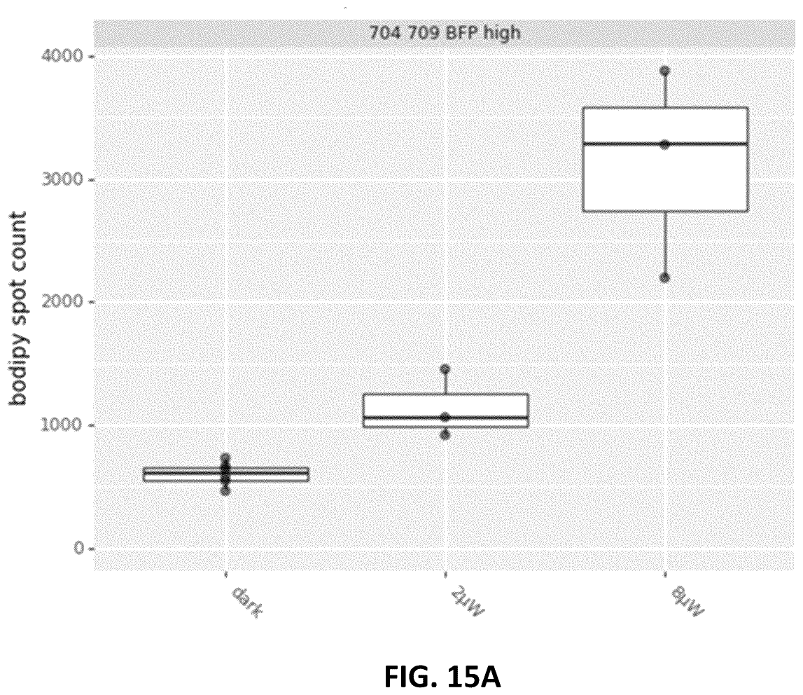

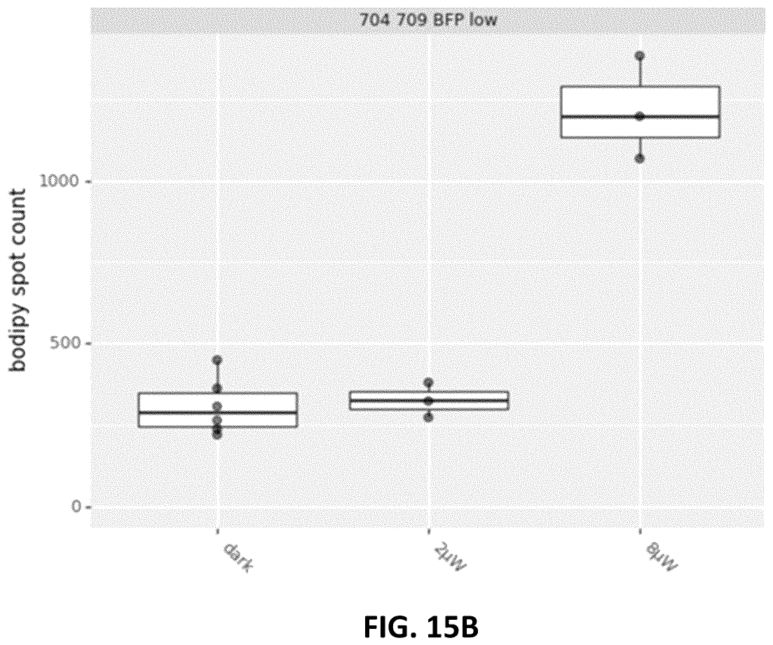

In this experiment, fibroblasts expressing SV40-LgT (SV40 cells) were stably transfected with plasmid 704 (a construct which enables PPARg+CEBPa expression upon recombination) using the PiggyBac system. Cells were selected with antibiotic so that the entire population expressed the plasmid 704. These cells were then stably transfected with plasmid 709 (a construct which drives constitutive expression of a light-activatable recombinase) using the Sleeping Beauty system. Cells expressing plasmids 704 and 709 were sorted into populations that had high expression of plasmid 709 and low expression of plasmid 709. Cells were handled under red or green light since room light activates the recombinase and permanently alters the DNA. Cells were then cultured in 2D and either exposed to dark conditions; 2 μW/mm² of blue light (465 nm) with a 20 seconds ON, 60 seconds OFF pulsing pattern; or 8 μW/mm² of blue light (465 mu) with a 20 seconds ON, 60 seconds OFF pulsing pattern. Light was turned off after 48 hours illumination, and media was exchanged for adipogenic media for 6 days. Cells were stained with BODIPY 493/503. As depicted in FIG. 14, increasing light power induced an increase in lipid droplet accumulation in both the cells with high expression of the light-activatable recombinase (FIG. 14, panels A, B, and C) and cells with low expression of the light-activatable recombinase (FIG. 14, panels D, E, and F). This data demonstrates that light induces differentiation of fibroblasts to fat cells in a power dependent manner. FIGS. 15A and 15B depict quantification of the results shown in FIG. 14.

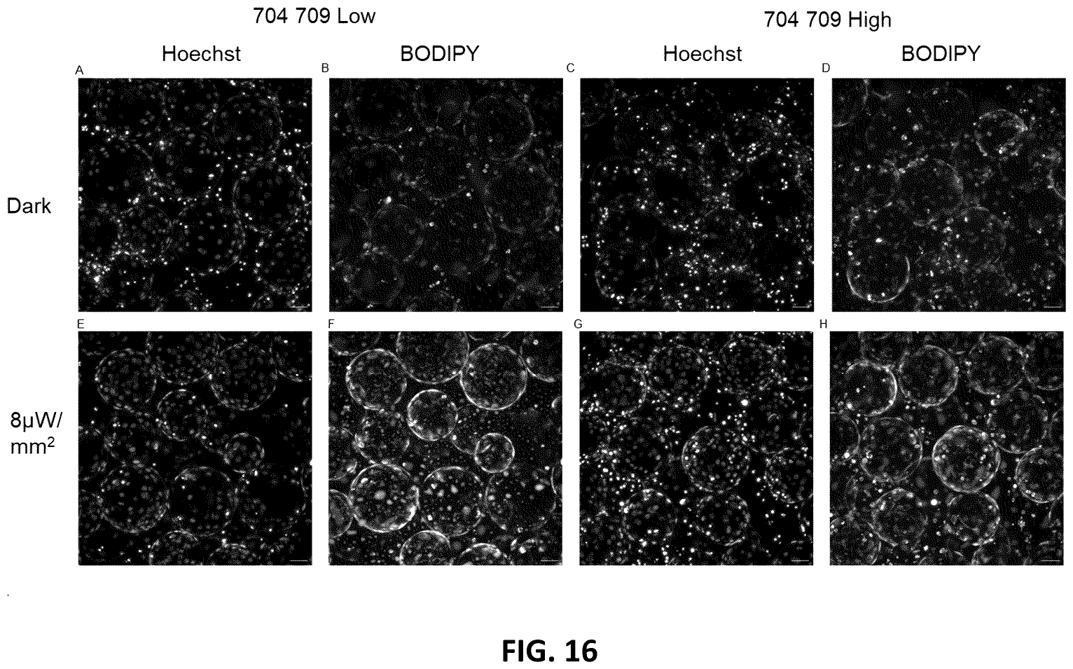

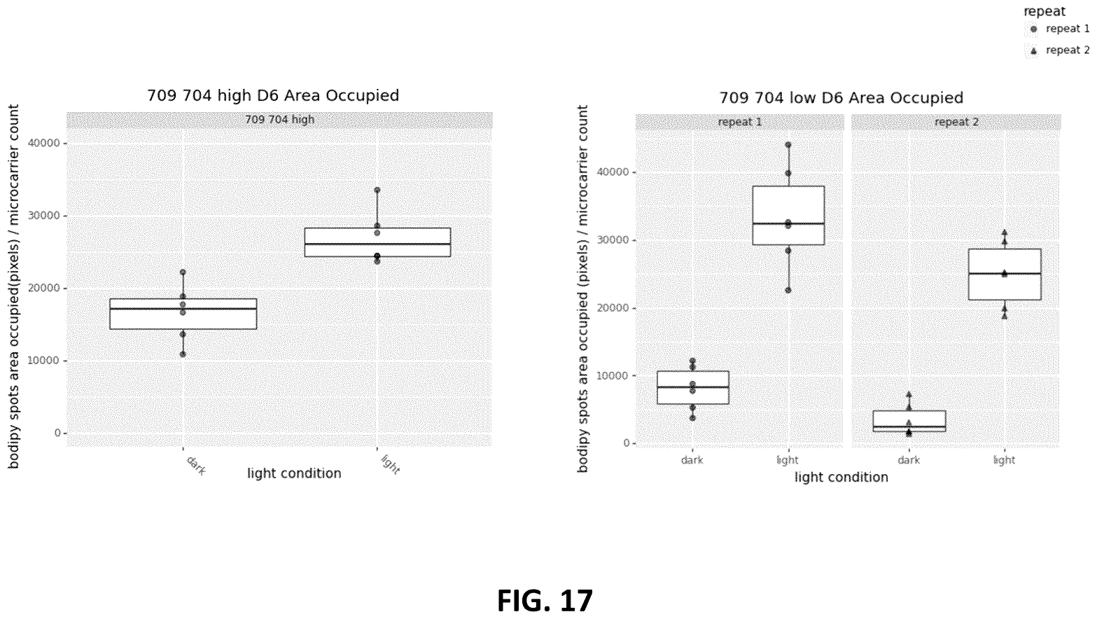

These results were further confirmed in three-dimensional suspension culture. In this experiment, fibroblasts expressing SV40 LgT (SV40 cells) were stably transfected with plasmid 704 using the PiggyBac system (a construct which enables PPARg+CEBPa expression upon recombination). Cells were selected with antibiotic so that the entire population expressed the plasmid 704. These cells were then stably transfected with plasmid 709 (a construct which drives constitutive expression of a light-activatable recombinase) using the Sleeping Beauty system. Cells expressing plasmids 704 and 709 were sorted into populations that had high expression of plasmid 709 and low expression of plasmid 709. Cells were handled under red or green light since room light activates the recombinase and permanently alters the DNA. Cells were attached to Cytodex 3 microcarriers in 100 ml growth media and cultured in spinner flasks. During this time, cells were either kept in the dark or illuminated with 8 μW/mm² of blue light (465 nm) with a 20 seconds ON, 60 seconds OFF pulsing pattern for 48 hours. After 48 hours, illumination was switched off and media was changed to 100 ml StemPro adipogenic media. The media was exchanged after 3 days. The cells+microcarriers were then stained with hoechst (stains nuclei) and BODIPY 493/503 (stains lipid droplets). As depicted in FIG. 16, illumination with blue light induced an increase in lipid droplet accumulation in both the cells with high expression of the light-activatable recombinase (FIG. 16, panel H) and cells with low expression of the light-activatable recombinase (FIG. 16, panel F). This data demonstrates that light can be used to induce differentiation of fibroblasts to fat cells in suspension culture. Images are z projections and representative images are shown in FIG. 16. The area covered by BODIPY 493/503stain, normalized to the number of microcarriers in the image was quantified and is shown in FIG. 17.

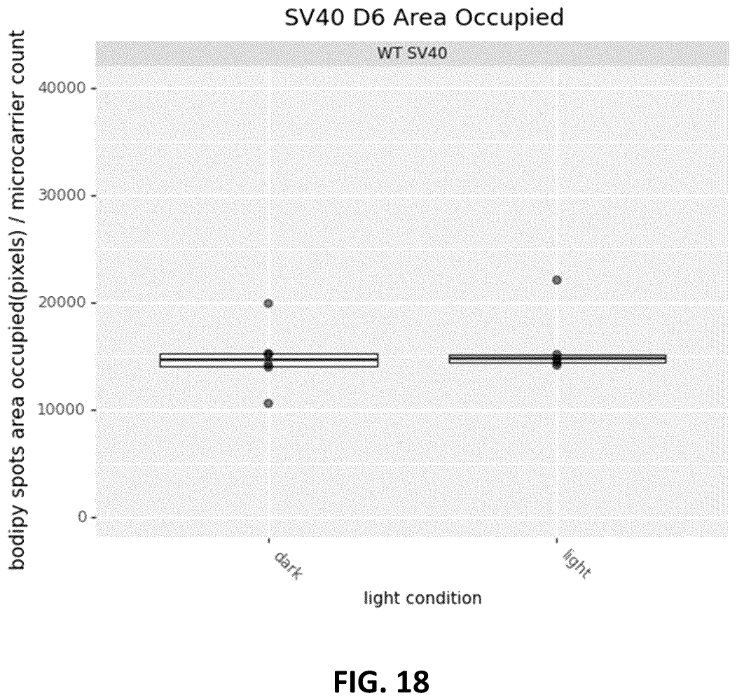

As a control, SV40 cells (without expression of the light-activatable recombinase or the adipogenic transcription factors) were attached to Cytodex 3 microcarriers in 100 ml growth media and cultured in spinner flasks. During this time, cells were illuminated with 8 μW/mm² of blue light (465 nm), with 20 seconds ON, 60 seconds OFF pulsing pattern for 48 hours. After 48 hours, illumination was switched off and media was changed to 100 ml StemPro adipogenic media. The media was exchanged after 3 days. The cells+microcarriers were then stained with hoechst (stains nuclei) and BODIPY 493/503 (stains lipid droplets). Images are z projections. The area covered by BODIPY 493/503 stain, normalized to the number of microcarriers in the image was quantified and is depicted in FIG. 18. This data demonstrates that illumination of SV40 cells with blue light alone, in the absence of expression of the light-activatable recombinase and the adipogenic transcription factors, does not increase lipid droplet accumulation and that expression of the light-activatable recombinase (at the appropriate expression level) and the constructs encoding the adipogenic transcription factors is needed.

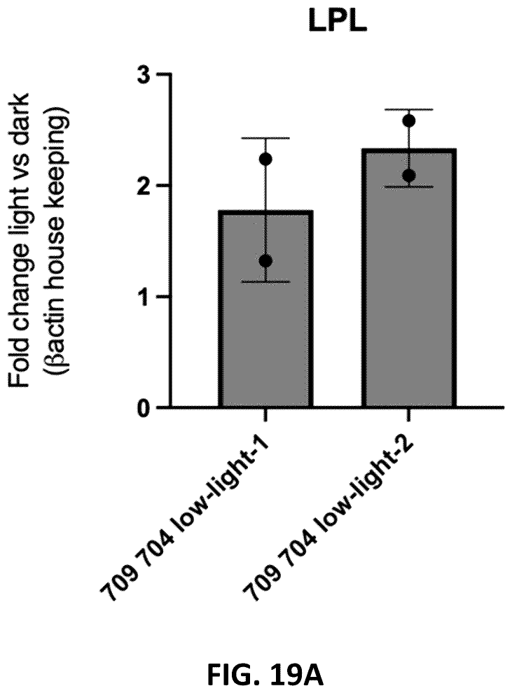

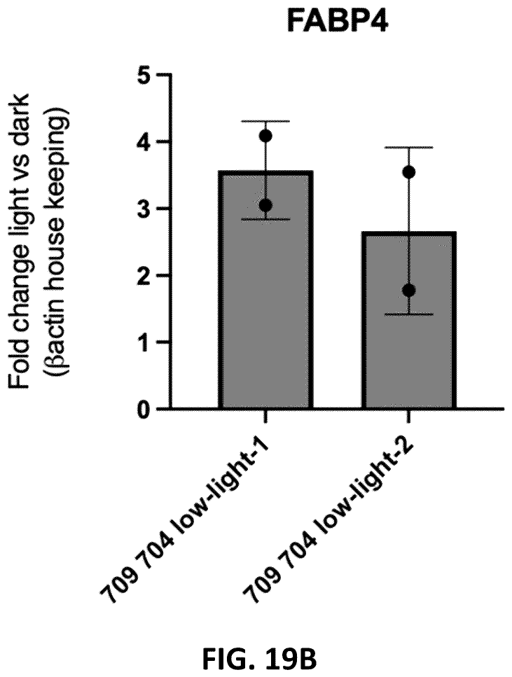

To demonstrate that genes associated with adipogenesis were expressed in cells induced to differentiate to adipocyte-like cells using light, samples from the cells depicted in FIG. 16, panels B and F, were taken. RNA was extracted and cDNA was generated. A qPCR was run to interrogate expression of two key adipogenesis genes, FABP4 and LPL. FABP4 encodes the fatty acid binding protein found in adipocytes and LPL encodes lipoprotein lipase, which is expressed in adipose tissue. FIGS. 19A and 19B demonstrate that adipocyte associated genes were upregulated in the light as compared to the dark, mirroring the increased lipid accumulation demonstrated in response to light as depicted in FIG. 16, panels B and F. Gene expression is shown in FIGS. 19A and 19B as the fold change of expression in the light versus the dark.

Taken together, these experiments demonstrated that 8 μW/mm² blue light activated the light-activatable recombinases which excised the blocking sequence, leading to PPARg/CEBPa expression and induced lipid accumulation demonstrating that light caused the fibroblasts to differentiate towards the adipocyte phenotype. They also demonstrated that the level of light-activatable recombinase expression was important for control of differentiation. The cells highly expressing the light-activatable recombinase demonstrated greater leaky light-activatable recombinase activation in the dark, leading to greater PPARg/CEBPa expression and lipid droplet accumulation in the dark compared to the cells with low light-activatable recombinase expression in the dark. This is likely because when the expression of the light-activatable recombinase is too high, the likelihood of the two halves of the light-activatable recombinase coming together increases to a point where the mechanism is less reliant on light. Hence, having an appropriate level of expression of the light-activatable recombinase is important for achieving control over the differentiation of fibroblasts towards adipocyte-like cells in suspension using light.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A method of differentiating at least one cell of a population of cells into a desired cell lineage, the method comprising:
(a) providing or obtaining the population of cells in a suspension culture; and
(b) controlling differentiation of the at least one cell of the population of cells with light, thereby differentiating the at least one cell into a desired cell lineage.

2. The method of claim 1, wherein the controlling differentiation of (b) comprises illuminating the at least one cell with light at a first wavelength or removing light at a first wavelength from the at least one cell.

3. (canceled)

4. The method of claim 1, wherein the at least one cell is genetically engineered to contain an exogenous nucleic acid comprising a nucleic acid sequence encoding for at least one transcription factor, at least one differentiation factor, or both, that effect differentiation into the desired cell lineage.

5. The method of claim 4, wherein the expression of the at least one transcription factor, the at least one differentiation factor, or both, is induced by the illuminating or the removing light.

6. The method of claim 5, wherein the exogenous nucleic acid comprises at least one promoter operably linked to the at least one transcription factor, the at least one differentiation factor, or both.

7. The method of claim 6, wherein the at least one cell is genetically engineered to contain an exogenous nucleic acid comprising a nucleic acid sequence encoding for at least one light-controllable transcriptional regulator.

8. The method of claim 7, wherein the promoter is an inducible promoter.

9. The method of claim 8, wherein the at least one light-controllable transcriptional regulator is a light-controllable transcriptional activator.

10. The method of claim 9, wherein the light-controllable transcriptional activator comprises a transcriptional activator fused to a light-controllable domain.

11. The method of claim 10, wherein the illuminating or the removing induces the light-controllable transcriptional activator to bind to and activate the inducible, thereby causing expression of the at least one transcription factor, the at least one differentiation factor, or both.

12. (canceled)

13. The method of claim 7, wherein the promoter is a constitutive promoter.

14. The method of claim 13, wherein the at least one light-controllable transcriptional regulator is a light-controllable transcriptional repressor.

15. The method of claim 14, wherein the light-controllable transcriptional repressor comprises a transcriptional repressor fused to a light-controllable domain.

16. The method of claim 15, wherein the illuminating induces the light-controllable transcriptional repressor to dissociate from the constitutive promoter, thereby causing expression of the at least one transcription factor, the at least one differentiation factor, or both.

17. The method of claim 15, wherein the removing light induces the light-controllable transcriptional repressor to dissociate from the constitutive promoter, thereby causing expression of the at least one transcription factor, the at least one differentiation factor, or both.

18. The method of claim 13, wherein the exogenous nucleic acid further comprises a blocking sequence downstream of the at least one promoter which, when present, blocks expression of the at least one first transcription factor, the at least one differentiation factor, or both.

19. The method of claim 18, wherein the at least one cell further comprises a nucleic acid sequence encoding at least one light-controllable recombinase.

20. The method of claim 19, wherein the at least one light-controllable recombinase is a light-activatable recombinase.

21. The method of claim 20, wherein the light-activatable recombinase comprises a recombinase or a portion thereof fused to a light-activatable domain.

22. The method of claim 18, wherein the blocking sequence is flanked by recombinase recognition sites that are recognized by the at least one light-controllable recombinase.

23. The method of claim 22, wherein the illuminating activates the light-controllable recombinase, thereby resulting in excision of the blocking sequence and inducing expression of the at least one transcription factor, the at least one differentiation factor, or both.

24. The method of claim 1, wherein the at least one cell is a stem cell, a pluripotent stem cell or a multipotent stem cell.

25. (canceled)

26. The method of claim 1, wherein the at least one cell is a fibroblast.

27. (canceled)

28. The method of claim 1, wherein the desired cell lineage is selected from the group consisting of: an adipocyte, a myocyte, and a chondrocyte.

29. The method of claim 4, wherein the at least one transcription factor is selected from the group consisting of: PPAR gamma, CEBP alpha, MYOD, MYOG, Myf5, MRF4, HEYL, KLF4, PAX3, SOX9, SOX5, SOX6, and any combination thereof.

30. The method of claim 2, wherein the illuminating further comprises illuminating a plurality of cells of the population of cells with light to differentiate each of the plurality of cells into the desired cell lineage; or the removing further comprises removing light from the population of cells to differentiate each of the plurality of cells into the desired cell lineage.

31-35. (canceled)

36. A system for differentiating a population of cells, the system comprising:
(a) a population of cells in suspension culture, wherein at least one cell of the population of cells is engineered to contain an exogenous nucleic acid comprising a nucleic acid sequence encoding for at least one transcription factor, at least one differentiation factor, or both, that effects differentiation into a desired cell lineage, wherein expression of the at least one transcription factor, the at least one differentiation factor, or both, is controlled by light; and
(b) one or more light source configured to illuminate at least one cell of the population of cells with light at a first wavelength.

37-65. (canceled)
